(19) United States
(12) Patent Application Publication
(10) Pub. No.: US 2022/0362530 A1
(43) Pub. Date: Nov. 17, 2022

(54) METHODS FOR TREATING NEOPLASTIC CONDITIONS OF THE SKIN

(71) Applicant: AQUAVIT PHARMACEUTICALS, INC., New York, NY (US)

(72) Inventor: Sobin Chang, New York, NY (US)

(21) Appl. No.: 17/763,017

(22) PCT Filed: Sep. 23, 2020
(86) PCT No.: PCT/US20/52283

§ 371 Date: Mar. 23, 2022

Related U.S. Application Data
(60) Provisional application No. 62/904,660, filed on Sep. 23, 2019.

(51) Int. Cl. A61M 37/00; A61K 45/06; A61K 9/00

(52) U.S. Cl. CPC A61M 37/0015; A61K 45/06; A61K 9/0021

(57) ABSTRACT

The present invention provides a method for treating a skin neoplasm in a subject, comprising administering to the subject's skin a composition comprising an effective amount of one or more antineoplastic agents, wherein the composition is administered with a microneedle delivery device.

### CROSS-REFERENCE TO RELATED APPLICATIONS

This application claims the benefit of U.S. Provisional Application No. 62/904,660, filed Sep. 23, 2019, the contents of which are incorporated by reference herein in their entirety.

### FIELD OF THE INVENTION

The field of the invention relates generally to the field of medicine, specifically methods and compositions useful for treating neoplastic conditions of the skin, such as actinic keratosis and basal cell carcinoma.

### BACKGROUND OF THE INVENTION

Skin neoplasms may occur in many different parts of the body. Symptoms associated with skin neoplasms include, e.g., changes in the color and/or appearance of the skin, such as the formation of a colored patch of skin, the formation of a new growth, and/or the formation of a sore or ulcer that resists healing. Symptoms may also include changes in existing skin structures, such as moles, and may include, e.g., changes in the border of the mole, such as formation of jagged edges around the mole, enlargement of the mole, and/or changes in the elevation of the mole.

Skin neoplasms can originate in any of the various layers of the skin, e.g., in the epidermis, dermis, or hypodermis. If left untreated, skin neoplasms can grow in size within a particular layer of the skin, and can also penetrate into other layers of the skin. Eventually, some of the cells in the skin neoplasm can break off from the original growth and enter the blood stream or the lymphatic system, allowing the skin neoplasm cells to travel to other parts of the body and form satellite tumors. Such tumors typically form in areas of the body with a high blood supply, such as the brain, bones, and liver. Once established, the satellite tumors can cause significant damage in these locations.

Skin neoplasms are one of the most prevalent diseases in the United States, with over 3.5 million cases diagnosed annually (Rogers, H W, Weinstock, M A, Harris, A R, et al. Incidence estimate of nonmelanoma skin cancer in the United States, 2006. Arch Dermatol 2010; 146(3):283-287). Melanoma, for example, is one of the more deadly forms of cancer, accounting for approximately 75% of deaths due to skin cancer (American Cancer Society, Cancer Facts and Figures 2010).

Treatment of skin neoplasms generally depends on the specific type of neoplasm as well as the location of the neoplasm on the patient's body, and may include surgical removal (e.g., curettage and desiccation, large-scale surgical removal of tumors), chemotherapy (e.g., topical administration of chemotherapeutic agents), cryotherapy (e.g., freezing and removing a tumor), and/or radiation therapy (e.g., external beam radiotherapy and/or brachytherapy).

One of the problems associated with current treatments relates to the difficulty of delivering therapeutically effective doses of the antineoplastic agent in a safe and effective manner to patients while minimizing side effects of the treatment. What is needed are new and effective methods to locally deliver effective amounts of antineoplastic agents to the skin to improve bioavailability while minimizing systemic effects and/or other side effects of treatment.

This background information is provided for informational purposes only. No admission is necessarily intended, nor should it be construed, that any of the preceding information constitutes prior art against the present invention.

### SUMMARY OF THE INVENTION

It is to be understood that both the foregoing general description of the embodiments and the following detailed description are exemplary, and thus do not restrict the scope of the embodiments.

In one aspect, the invention provides method for treating a skin neoplasm in a subject, comprising administering to the subject's skin a composition comprising an effective amount of one or more antineoplastic agents, wherein the composition is administered with a microneedle delivery device.

In another aspect, the invention provides a microneedle delivery device comprising an effective amount of one or more antineoplastic agents for use in the methods herein.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE FIGURES

The skilled artisan will understand that the drawings, described below, are for illustration purposes only. The drawings are not intended to limit the scope of the present teachings in any way.

FIG. 1 is a view of a handheld microneedle injection apparatus. The syringe ejection volume is automatically controlled and dispenses into an interchangeable head containing one or several needles. The diagram shows the connection of corrugated connector and microneedle head. The rubber based connector is such that its flexibility will allow connections with small openings (1) and large ones (2) to fit and seal the microneedle head. The corrugated connector, also made of rubber (3), will further allow larger embodiments to connect to this system with the spring plate microneedle head (4).

FIG. 2 is an image of a microneedle head piece.

FIG. 3 is a schematic representation of a device in a syringe configuration. Alternative configurations include vial- and capsule-loaded configurations. The device holds a syringe (2) for automatic injection via a plurality of microneedles in the microneedle head. Ejection volume is controlled by an information processor (9). Other elements are noted: the motor or actuator (4) to control the piston (3), exchangeable and controllable needle head (1) and cam system and dial to adjust needle injection depth (5), and needle head ejector (10). Information is shown to the user in a display panel that may include a manual or touchscreen control panel (12) and data is stored in a storage unit (11) that may be removable. The needle head (1) may be controlled by an actuator (13).

FIG. 4 provide three additional views of a microneedle device. Microneedle components: (A) microneedles, (B) housing of the needles and (C) a reservoir.

FIG. 5 provides an exemplary microneedle drug delivery device.

FIG. 6 provides an exemplary microneedle drug delivery device.

FIG. 7 provides internal assembly of parts of the device of FIG. 6.

FIG. 8 provides an external push assembly view of the device of FIG. 6.

FIG. 9 provides a view of the assembled internal parts of FIG. 6.

FIG. 10 provides a view of the assembled internal parts of FIG. 6.

FIG. 11 provides a view of the device of FIG. 6.

FIG. 12 provides a view of the device of FIG. 6.

FIG. 13 illustrates a multi-chamber microneedle drug delivery device design that features a pusher that is activated by the subject. The pusher pierces the layer separating Chamber I and Chamber II thereby allowing flow of bioactive composition from chamber I to chamber II. After this, the bioactive compositions are mixed by a gravity-driven motion by shaking the device. After this the bioactive composition transfers to the reservoir, and can be administered on a subject. The microchannel head facilitates movement from reservoir to the subject's skin.

FIG. 14 illustrates a multi-chamber microneedle drug delivery device design that features a pusher that is activated by the subject. The pusher pierces the layer separating Chamber I and Chamber II thereby allowing flow of bioactive composition from chamber I to chamber II. After this, the bioactive compositions are mixed by a gravity-driven motion by shaking the device. After this the bioactive composition transfers to the reservoir, and can be administered on a subject. The microchannel head facilitates movement from reservoir to the subject's skin.

FIG. 15 illustrates a modular multi-chamber microneedle drug delivery device design. This allows the chambers and the reservoir with the microneedle head to be detachable. The chambers can be replaced or substituted. It features a pusher that is activated by the subject. The pusher pierces the layer separating Chamber I and Chamber II thereby allowing flow of bioactive composition from chamber I to chamber II. After this, the bioactive compositions are mixed by a gravity-driven motion by shaking the device. After this the bioactive composition transfers to the reservoir and can be administered on a subject. The microchannel head facilitates movement from reservoir to the subject's skin.

FIG. 16 illustrates a multi-chamber microneedle drug delivery device design that features a pusher that is activated by the subject. The pusher pierces the layer separating Chamber I and Chamber II thereby allowing flow of bioactive composition from chamber I to chamber II. After this, the bioactive compositions are mixed by a gravity-driven motion by shaking the device. After this the bioactive composition transfers to the reservoir and can be administered on a subject. The microchannel head facilitates movement from reservoir to the subject's skin. It also features a blender that can be activated by the subject through an external button/switch. This blender helps in mixing the bioactive composition.

FIG. 17 illustrates a multi-chamber microneedle drug delivery device design that features multiple pusher that is activated individually or together by the subject. Each pusher pierces the layer separating the two chambers thereby allowing flow of bioactive composition from one chamber to another. After this, the bioactive compositions are mixed by a gravity-driven motion by shaking the device. After this the bioactive composition transfers to the reservoir and can be administered on a subject. The microchannel head facilitates movement from reservoir to the subject's skin. Each of these chambers can contain different compositions.

FIG. 18 illustrates a modular multi-chamber microneedle drug delivery device design that features multiple chambers that can be attached to each other. Each chamber features a pusher that pierces the layer separating the two chambers thereby allowing flow of bioactive composition from one chamber to another. After this, the bioactive compositions are mixed by a gravity-driven motion by shaking the device. After this the bioactive composition transfers to the reservoir, and can be administered on a subject. The microchannel head facilitates movement from reservoir to the subject's skin. Each of these chambers can contain different compositions.

FIG. 19 illustrates a modular multi-chamber microneedle drug delivery device design that features two chambers that can be attached to each other wherein one chamber contains the pusher that pierces the other chamber. The pusher pierces the outer layer of the attached chamber thereby allowing flow of bioactive composition from one chamber to another. After this, the bioactive compositions are mixed by a gravity-driven motion by shaking the device. After this the bioactive composition transfers to the reservoir and can be administered on a subject. The microchannel head facilitates movement from reservoir to the subject's skin. Each of these chambers can contain different compositions.

FIG. 20 illustrates a microchannel head adapter that can be used with regular syringes. It comes with a cap that covers the microchannel head.

### DETAILED DESCRIPTION OF THE INVENTION

Reference will now be made in detail to the presently preferred embodiments of the invention which, together with the drawings and the following examples, serve to explain the principles of the invention. These embodiments describe in sufficient detail to enable those skilled in the art to practice the invention, and it is understood that other embodiments may be utilized, and that structural, biological, and chemical changes may be made without departing from the spirit and scope of the present invention. Unless defined otherwise, all technical and scientific terms used herein have the same meanings as commonly understood by one of ordinary skill in the art.

Unless defined otherwise, all technical and scientific terms used herein have the same meaning as commonly understood by those of ordinary skill in the art to which this invention pertains. The following references provide one of skill with a general definition of many of the terms used in this invention: Academic Press Dictionary of Science and Technology, Morris (Ed.), Academic Press (1^{st} ed., 1992); Oxford Dictionary of Biochemistry and Molecular Biology, Smith et al. (Eds.), Oxford University Press (revised ed., 2000); Encyclopaedic Dictionary of Chemistry, Kumar (Ed.), Anmol Publications Pvt. Ltd. (2002); Dictionary of Microbiology and Molecular Biology, Singleton et al. (Eds.), John Wiley & Sons (3rd ed., 2002); Dictionary of Chemistry, Hunt (Ed.), Routledge (1^{st} ed., 1999); Dictionary of Pharmaceutical Medicine, Nahler (Ed.), Springer-Verlag Telos (1994); Dictionary of Organic Chemistry, Kumar and Anandand (Eds.), Anmol Publications Pvt. Ltd. (2002); and A Dictionary of Biology (Oxford Paperback Reference), Martin and Hine (Eds.), Oxford University Press (4^{th} ed., 2000). Further clarifications of some of these terms as they apply specifically to this invention are provided herein.

For the purpose of interpreting this specification, the following definitions will apply and whenever appropriate, terms used in the singular will also include the plural and vice versa. In the event that any definition set forth below conflicts with the usage of that word in any other document, including any document incorporated herein by reference, the definition set forth below shall always control for purposes of interpreting this specification and its associated claims unless a contrary meaning is clearly intended (for example in the document where the term is originally used). The use of “or” means “and/or” unless stated otherwise. As used in the specification and claims, the singular form “a,” “an” and “the” include plural references unless the context clearly dictates otherwise. For example, the term “a cell” includes a plurality of cells, including mixtures thereof. The use of “comprise,” “comprises,” “comprising,” “include,” “includes,” and “including” are interchangeable and not intended to be limiting. Furthermore, where the description of one or more embodiments uses the term “comprising,” those skilled in the art would understand that, in some specific instances, the embodiment or embodiments can be alternatively described using the language “consisting essentially of” and/or “consisting of.”

One skilled in the art may refer to general reference texts for detailed descriptions of known techniques discussed herein or equivalent techniques. These texts include Current Protocols in Molecular Biology (Ausubel et. al., eds. John Wiley & Sons, N.Y. and supplements thereto), Current Protocols in Immunology (Coligan et al., eds., John Wiley St Sons, N.Y. and supplements thereto), Current Protocols in Pharmacology (Enna et al., eds. John Wiley & Sons, N.Y. and supplements thereto) and Remington: The Science and Practice of Pharmacy (Lippincott Williams & Wilicins, 2Vt edition (2005)), for example.

It is contemplated that any method or composition described herein can be implemented with respect to any other method or composition described herein.

As used herein, the term “about” means plus or minus 10% of the numerical value of the number with which it is being used.

The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A. B, and C; A, B. or C; A or C; A or B; B or C; A and C; A and B; B and C; A (alone), B (alone); and C (alone).

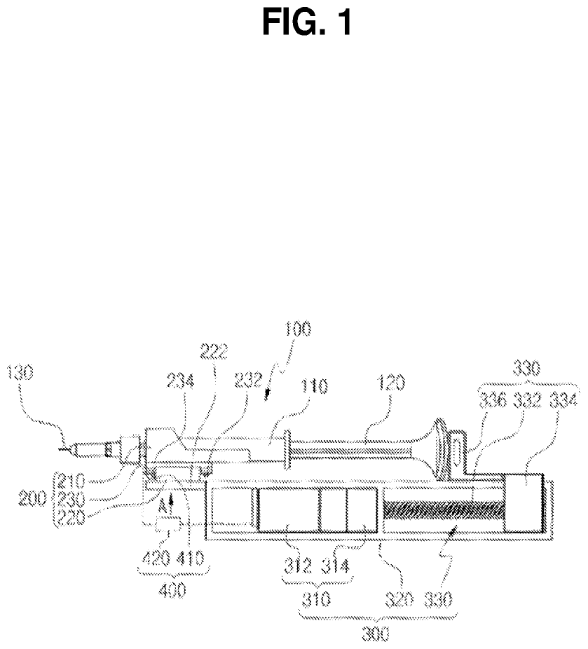

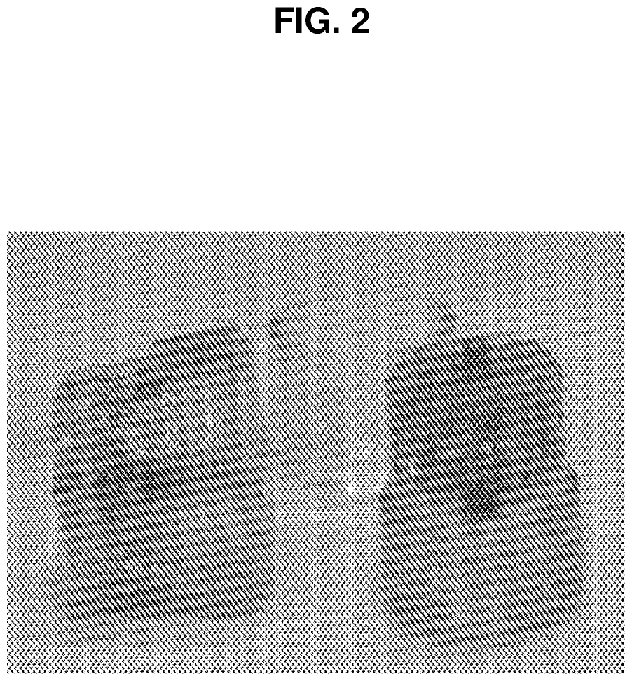

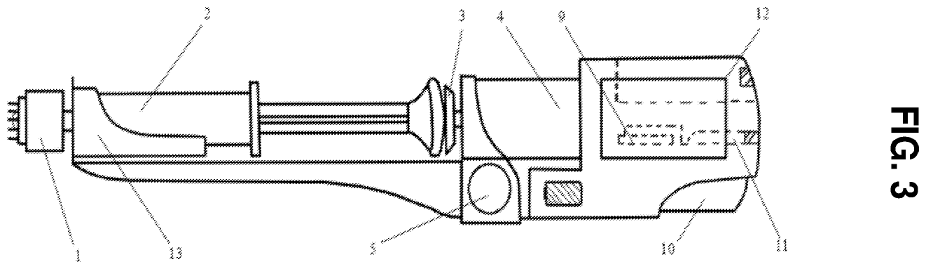

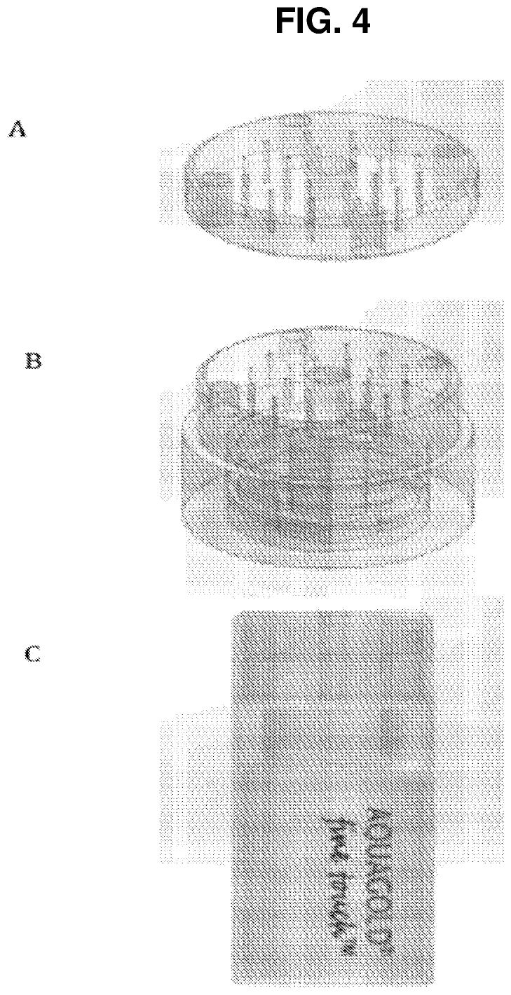

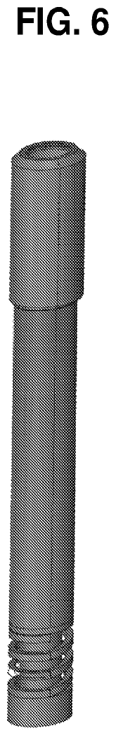

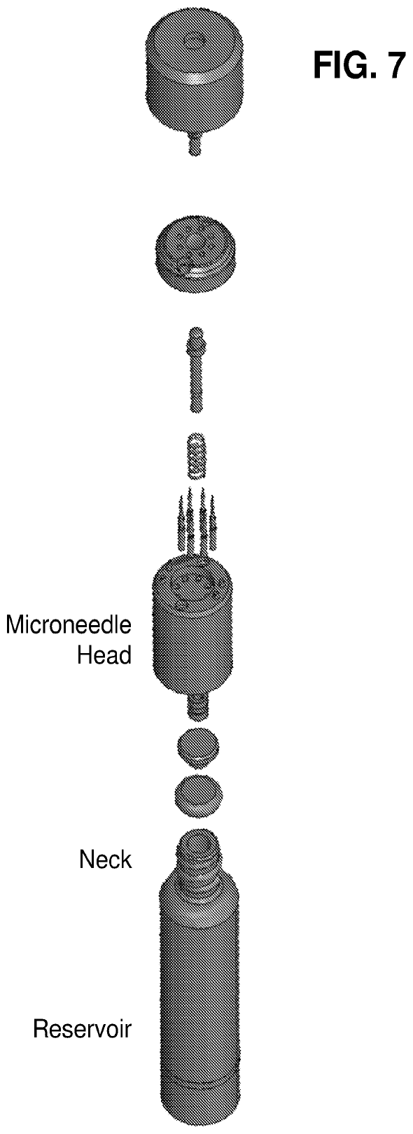

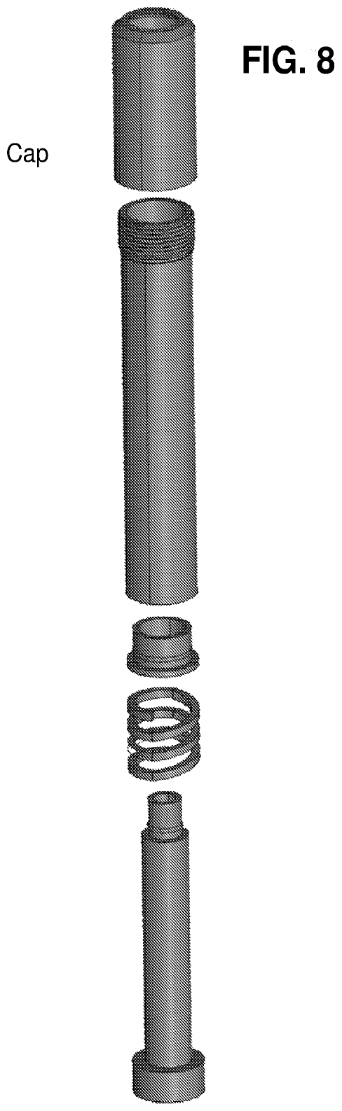

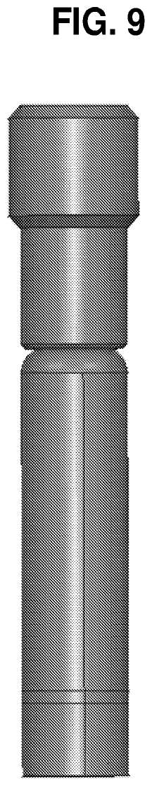

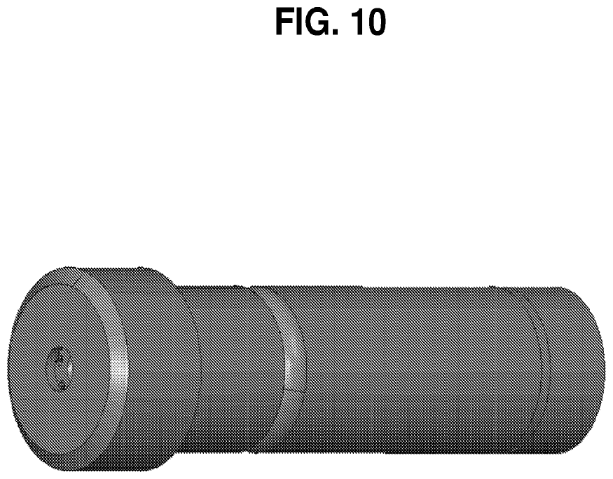

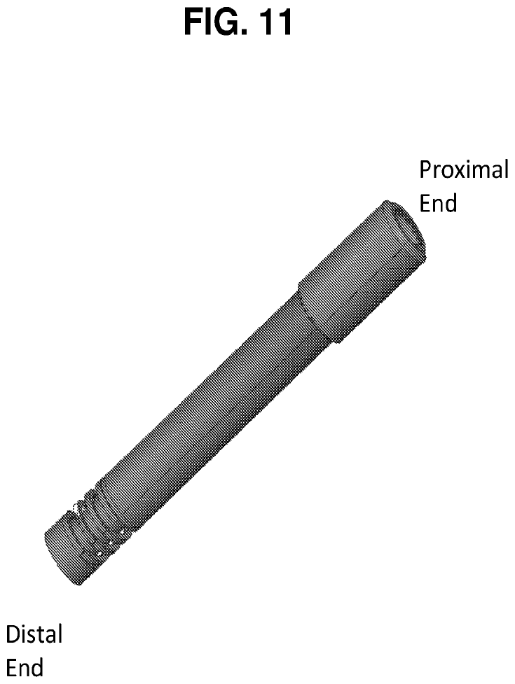

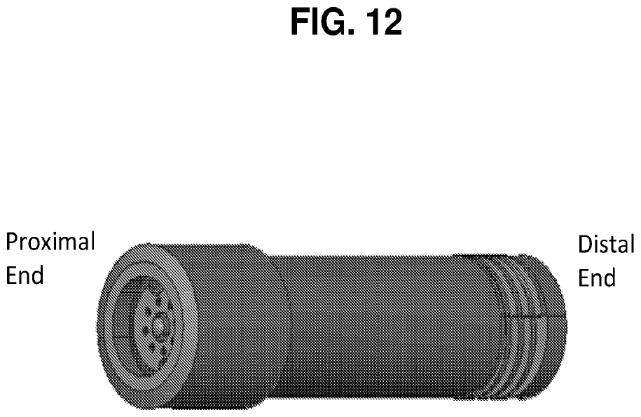

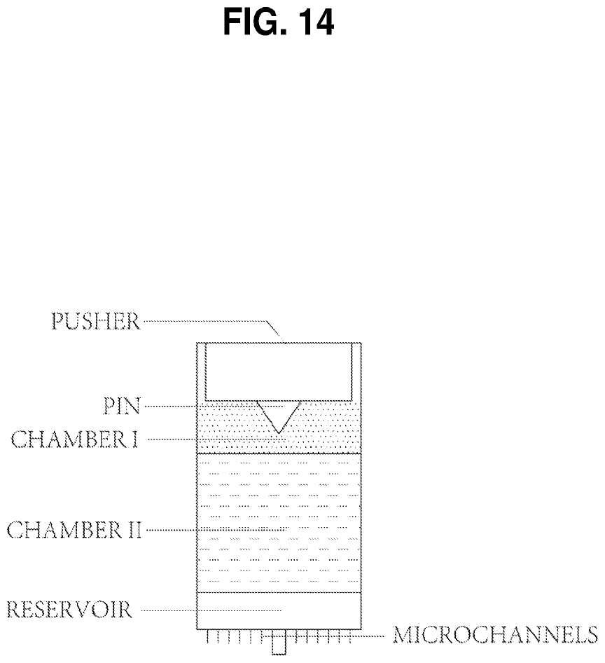

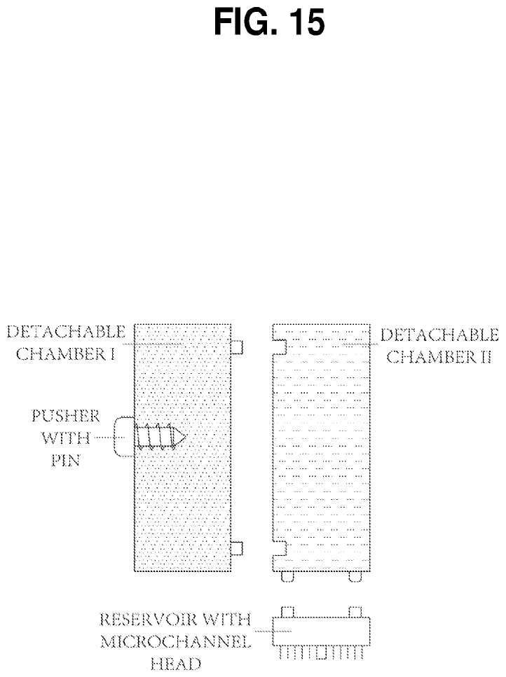

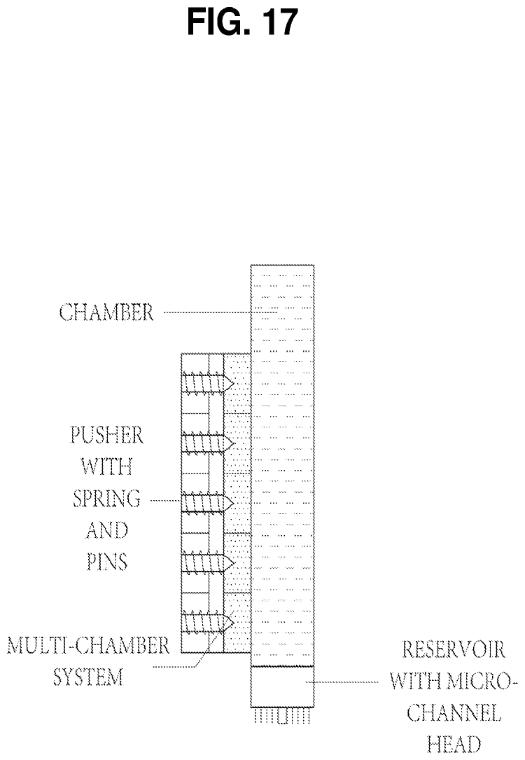

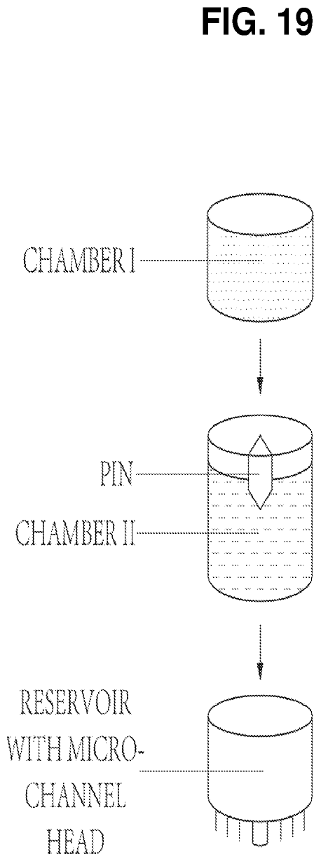

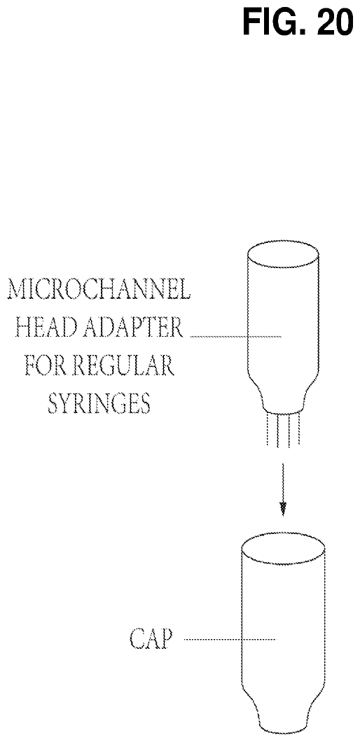

In one embodiment, the invention provides a method for treating a skin neoplasm in a subject, comprising administering to the subject's skin a composition comprising an effective amount of one or more antineoplastic agents, wherein the composition is administered with a microneedle delivery device. In some embodiments, the microneedle delivery device useful in the methods of the invention is depicted in any of FIGS. 1-20.

As provided herein, a microneedle delivery device is used to deliver the antineoplastic composition. In some embodiments, a microneedle array can be used to deliver the antineoplastic composition directly to the dermis (the second layer of skin). In some embodiments, the microneedle devices as disclosed herein deliver the antineoplastic composition into the dermal and epidermal junction area. In another embodiment, the microneedle device does not penetrate into the dermal layer but only disrupts the superficial portion of the skin, referred to as stratum corneum.

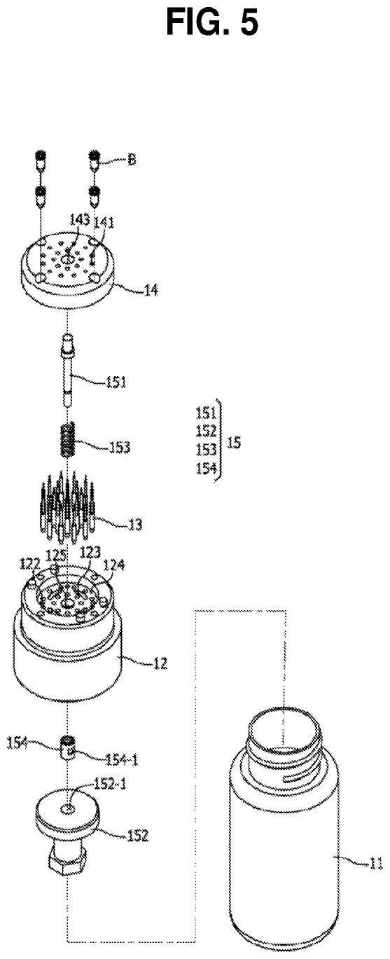

In some embodiments, the microneedle delivery device useful in the methods of the invention is depicted in FIG. 5. In some embodiments, the microneedle drug delivery device is as described in Korean Patent No. 10-1582822, which is incorporated by reference herein in its entirety. In some embodiments, the microneedle device useful in the methods of the invention is depicted in any of FIGS. 1-20.

In some embodiments, the microneedle device that can be used comprises multiple chambers. In some embodiments, the device comprises a plurality of modular or replaceable chambers, wherein the chambers can hold the antineoplastic compounds or compositions. In some embodiments, the multi-chamber device comprises one or more microneedles, wherein the microneedles are hollow or non-hollow, wherein one or multiple grooves are inset along an outer wall of the microneedles. In some embodiments, the multi-chamber device comprises a chamber that serves as a reservoir that holds the composition to be delivered, wherein the reservoir is attached to or contains a means to encourage flow of the antineoplastic composition contained in the reservoir into the skin of a subject. In some embodiments, the chambers can hold an antineoplastic compound or composition in a powder form or in an aqueous solution.

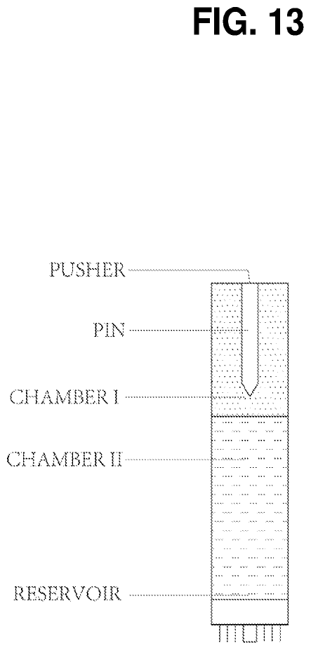

In some embodiments, the device comprises a chamber that comprises a pin that punctures another chamber to allow flow of contents from one of the chambers into the other chamber. See, for example, FIGS. 13-19.

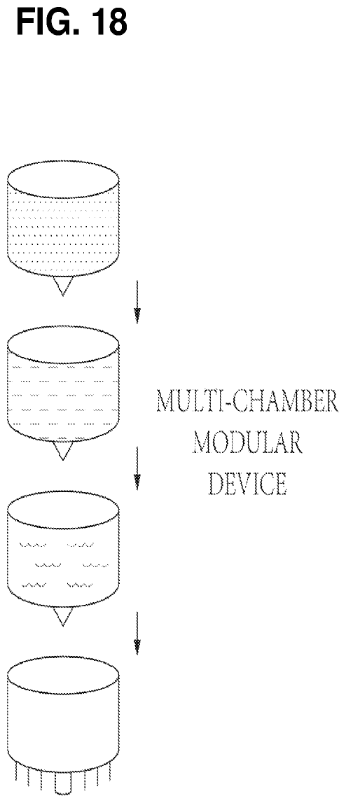

In some embodiments, the multi chamber microchannel delivery device is modular as described in FIG. 18. In some embodiments, each chamber of the device can be removed and added to the device through a push pin, mechanical or magnetic fittings.

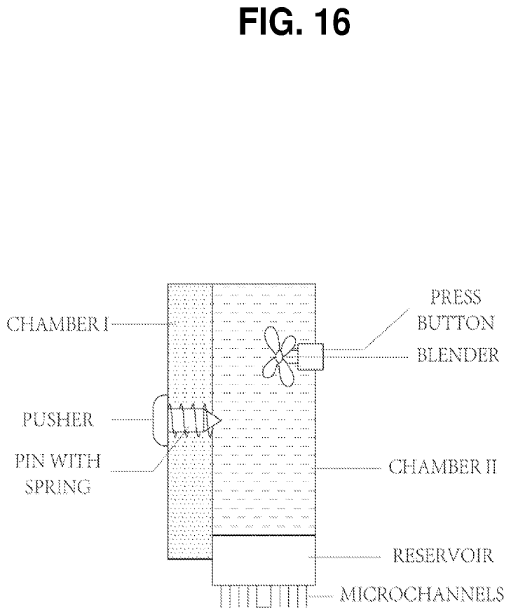

In some embodiments, the chamber contains a means for mixing the components, such as a blender element as shown in FIG. 16.

In some embodiments, the lining between the chambers are made of plastic films with low puncture resistance. In some embodiments, the lining between the chambers are made of deformable, preferably elastic material.

In some embodiments, the microneedle delivery device comprises
i) one or more microneedles, wherein the microneedles are hollow or non-hollow, wherein one or multiple grooves are inset along an outer wall of the microneedles; and
ii) a reservoir that holds the composition to be delivered, wherein the reservoir is attached to or contains a means to encourage flow of the bioactive composition contained in the reservoir into the skin.

In some embodiments, the composition is administered by the microneedle delivery device with a repeated motion of penetrating the microneedle delivery device into the skin of the subject. In some embodiments, the composition is delivered into the skin by passing through the one or multiple grooves along the outer wall of the microneedle. In some embodiments, the microneedles are non-hollow.

In some embodiments, the means to encourage flow of the composition contained in the reservoir into the skin is selected from the group consisting of a plunger, pump and suction mechanism. In some embodiments, the means to encourage flow of the composition contained in the reservoir into the skin is a mechanical spring loaded pump system.

In some embodiments, the microneedles have a single groove inset along the outer wall of the microneedle, wherein the single groove has a screw thread shape going clockwise or counterclockwise around the microneedle.

In some embodiments, the microneedles are from 0.1 mm to about 2.5 mm in length and from 0.01 mm to about 0.05 mm in diameter.

In some embodiments, the microneedles are made from a substance comprising gold.

In some embodiments, the one or more microneedles comprises an array of microneedles in the shape of a circle.

In some embodiments, the microneedles are made of 24-carat gold plated stainless steel and comprise an array of about 10 to about 50 microneedles. In some embodiments, the array comprises 20 microneedles.

In some embodiments, the microneedle delivery device is repeatedly pressed against the subject's skin to deliver the composition to the area of the skin to be treated. In some embodiments, the microneedle delivery device is repeatedly pressed about 10, about 20, about 30, about 40, about 50, about 100, about 200, about 300, about 400, about 500, about 600, about 700, about 800, about 900, about 1000, about 1100, about 1200, about 1300, about 1400, about 1500, about 1600, about 1700, about 1800, about 1900, or about 2000 or more times to administer the composition.

In some embodiments, the microneedle delivery device comprises a single or an array of microneedles. In some embodiments, the microneedles will have one or multiple grooves inset along its outer wall. This structural feature of the dermal delivery device allows liquids stored in a reservoir at the base of each needle to travel along the needle shaft into the tissue.

In some embodiments, the microneedle array comprises from about 1 to about 500 microneedles, which will be anywhere from about 0.1 to about 2.5 mm in length and from 0.01 to about 0.5 mm in diameter, and be composed of any metal, metal alloy, metalloid, polymer, or combination thereof, such as gold, steel, silicon, PVP (polyvinylpyrrlidone), etc. The microneedles will each have one or more recesses running a certain depth into the outer wall to allow for flow of the substance to be delivered down the microneedle and into the dermis; these recesses can be in a plurality of shapes, including but not limited to: straight line, cross shape (+), flat shape (−), or screw thread shape going clockwise or counterclockwise. The array will be in any shape or combination of shapes, continuous, or discontinuous. The list of possible shapes includes, but is not limited to, circles, triangles, rectangles, squares, rhomboids, trapezoids, and any other regular or irregular polygons. The array can be attached to a reservoir to hold the substances to be delivered, and this reservoir will be any volume (0.25 mL to 5 mL), shape, color, or material (glass, metal, alloy, or polymer), as determined necessary. This reservoir will itself be attached to or contain a means to encourage flow of the drug solutions contained in the reservoir into the skin. Two non-limiting examples of such means are 1) a plate and spring that allows the contained solutions to flow only when the device is tapped into the skin, and 2) a syringe that contains the drug solutions to be delivered and includes a plunger that can be depressed to mechanically drive the solution into the skin.

The microneedle delivery device is capable of delivering compositions directly to the epidermal, dermal and subcuticular layers of the skin. Therefore, it should be understood that further embodiments developed for use with non-hollow or hollow microneedle systems of delivery by those skilled in the art fall within the spirit and scope of this disclosure.

In another aspect, a microneedle device for use in the methods described herein is a device such as described in U.S. Pat. No. 8,257,324, which is hereby incorporated by reference. Briefly, the devices include a substrate to which a plurality of hollow microneedles are attached or integrated, and at least one reservoir, containing a bioactive formulation, selectably in communication with the microneedles, wherein the volume or amount of composition to be delivered can be selectively altered. The reservoir can be, for example, formed of a deformable, preferably elastic, material. The device typically includes a means, such as a plunger, for compressing the reservoir to drive the bioactive formulation from the reservoir through the microneedles, A reservoir, can be, for example, a syringe or pump connected to the substrate. A device, in some instances, comprises: a plurality of hollow microneedles (each having a base end and a tip), with at least one hollow pathway disposed at or between the base end and the tip, wherein the microneedles comprise a metal; a substrate to which the base ends of the microneedles are attached or integrated; at least one reservoir in which the material is disposed and which is in connection with the base end of at least one of the microneedles, either integrally or separably; a sealing mechanism interposed between the at least one reservoir and the substrate, wherein the sealing mechanism comprises a fracturable barrier; and a device that expels the material in the reservoir into the base end of at least one of the microneedles and into the skin. The reservoir comprises a syringe secured to the substrate, and the device that expels the material comprises a plunger connected to a top surface of the reservoir. The substrate may be adapted to removably connect to a standard or Luer-lock syringe. In one instance, the device may further include a spring engaged with the plunger. In another instance, the device may further include an attachment mechanism that secures the syringe to the device. In another instance, the device may further include a sealing mechanism that is secured to the tips of the microneedles. In another instance, the device may further include means for providing feedback to indicate that delivery of the material from the reservoir has been initiated or completed. An osmotic pump may be included to expel the material from the reservoir. A plurality of microneedles may be disposed at an angle other than perpendicular to the substrate.

In certain instances, the at least one reservoir comprises multiple reservoirs that can be connected to or are in communication with each other. The multiple reservoirs may comprise a first reservoir and a second reservoir, wherein the first reservoir contains a solid formulation and the second reservoir contains a liquid carrier for the solid formulation. A fracturable barrier for use in the devices can be, for example, a thin foil, a polymer, a laminate film, or a biodegradable polymer. The device may further comprise, in some instances, means for providing feedback to indicate that the microneedles have penetrated the skin.

In some embodiments, the device can include, in some instances, a single or plurality of solid, screw-type microneedles, of single or varied length. Typically the needles attach to a substrate or are embedded within the substrate. The substrate can be made of any metal, metal alloy, ceramics, organics metalloid, polymer, or combination thereof, including composites, such as gold, steel, silicon, PVP (polyvinylpyrrlidone) etc. The screw-shape dimensions may be variable. For example, in one embodiment the screw-shape may be a tight coiled screw shape, whereas in another embodiment the screw-shape might be a loose coiled screw shape whereby the screw threads in one embodiment lie closely together along the outer edge of the needle and, in another embodiment, the screw threads lie far from each other along the outer edge of the needle.

In one embodiment a reservoir would attach to the substrate to allow drug solution to flow down the side of the microneedles. In one embodiment the reservoir is a solid canister of differing sizes depending on the desired volume or amount of drug to be delivered. The reservoir contains the drug to be delivered. In another embodiment, the reservoir can be supported by a mechanical (spring loaded or electrified machine-driven) pump system to deliver the drug solution. In another embodiment, the reservoir is composed of a rubber, elastic, or otherwise deformable and flexible material to allow manual squeezing to deliver the drug solution. In another embodiment the device includes hollow needles or needles with alternative ridges and shapes to more efficiently drive solution from the reservoir through to the dermis.

A device described herein may contain, in certain instances, about twenty screw thread design surgical grade microneedles. Each microneedle has a diameter that is thinner than a human hair and may be used for direct dermal application. In one instance, a microneedle has a diameter of less than about 0.18 mm. In another instance, a microneedle has a diameter of about 0.15 mm, about 0.14 mm, about 0.13 mm, about 0.12 mm, about 0.11 mm, or about 0.10 mm. Each microneedle may be plated with 24 carat gold. The device allows for targeted and uniform delivery of a composition comprising an antineoplastic agent into the skin in a process that is painless compared to injectables. Administration can result in easy and precise delivery of a composition comprising an antineoplastic agent with generally no bruising, pain, swelling and bleeding. Delivery of an antineoplastic agent may include sensitive areas and areas difficult to treat with traditional methods, such as around the eyes and mouth.

The device may include means, manual or mechanical, for compressing the reservoir, creating a vacuum, or otherwise using gravity or pressure to drive the antineoplastic agent from the reservoir through the microneedles or down along the sides of the microneedle. The means can include a plunger, pump or suction mechanism. In another embodiment, the reservoir further includes a means for controlling rate and precise quantity of drug delivered by utilizing a semi-permeable membrane, to regulate the rate or extent of drug which flows along the shaft of the microneedles. The microneedle device enhances transportation of drugs across or into the tissue at a useful rate. For example, the microneedle device must be capable of delivering drug at a rate sufficient to be therapeutically useful. The rate of delivery of the drug composition can be controlled by altering one or more of several design variables. For example, the amount of material flowing through the needles can be controlled by manipulating the effective hydrodynamic conductivity (the volumetric through-capacity) of a single device array, for example, by using more or fewer microneedles, by increasing or decreasing the number or diameter of the bores in the microneedles, or by filling at least some of the microneedle bores with a diffusion-limiting material. It can be preferred, however, to simplify the manufacturing process by limiting the needle design to two or three “sizes” of microneedle arrays to accommodate, for example small, medium, and large volumetric flows, for which the delivery rate is controlled by other means.

Other means for controlling the rate of delivery include varying the driving force applied to the drug composition in the reservoir. For example, in passive diffusion systems, the concentration of drug in the reservoir can be increased to increase the rate of mass transfer. In active systems, for example, the pressure applied to the reservoir can be varied, such as by varying the spring constant or number of springs or elastic bands. In either active or passive systems, the barrier material can be selected to provide a particular rate of diffusion for the drug molecules being delivered through the barrier at the needle inlet.

The array may be in any shape or combination of shapes, continuous, or discontinuous. The list of possible shapes includes, but is not limited to, circles, triangles, rectangles, squares, rhomboids, trapezoids, and any other regular or irregular polygons.

The array may be attached to a reservoir to hold the substances to be delivered, and this reservoir may be any volume (about 0.25 mL to about 5 mL), shape, color, or material (glass, metal, alloy, or polymer), as determined necessary.

This reservoir can itself be attached to or contain a means to encourage flow of the drug solutions contained in the reservoir into the skin. Two non-limiting examples of such means are 1) a plate and spring that allows the contained solutions to flow only when the device is tapped into the skin, and 2) a syringe that contains the drug solutions to be delivered and includes a plunger that can be depressed to mechanically drive the solution into the skin.

In some embodiments, the device can include a single or plurality of solid, screw-type microneedles, of single or varied lengths housed in a plastic or polymer composite head which embodies a corrugated rubber connector. In some embodiments, the needles attach to a substrate or are embedded within the substrate. The substrate can be made of any metal, metal alloy, ceramics, organics metalloid, polymer, or combination thereof, including composites, such as gold, steel, silicon, PVP (polyvinylpyrrlidone) etc. The screw-shape dimensions may be variable. For example, in one embodiment the screw-shape may be a tight coiled screw shape, whereas in another embodiment the screw-shape might be a loose coiled screw shape. The corrugated rubber connector is a unique advantage conferring feature which bestows the microneedle head with a universally adoptable feature for interfacing the micro needle cartridges with multiple glass and or plastic vials, reservoirs and containers as well as electronic appendages for an altogether enhanced adjunct liquid handling, security and surveillance utility.

In one embodiment a reservoir would attach to the substrate to allow drug solution to flow down the side of the microneedles. In one embodiment the reservoir is a solid canister of differing sizes depending on the desired volume or amount of drug to be delivered. The reservoir contains the drug to be delivered. In another embodiment, the reservoir can be supported by a mechanical (spring loaded or electrified machine-driven) pump system to deliver the drug solution. In another embodiment, the reservoir is composed of a rubber, elastic, or otherwise deformable and flexible material to allow manual squeezing to deliver the drug solution. In another embodiment the device includes hollow needles or needles with alternative ridges and shapes to more efficiently drive solution from the reservoir through to the dermis.

A microneedle array can consist of from about 1 to about 500 microneedles, which will be anywhere from about 0.1 to about 2.5 mm in length and from 0.01 to about 0.5 mm in diameter, and be composed of any metal, metal alloy, metalloid, polymer, or combination thereof, such as gold, steel, silicon, PVP (polyvinylpyrrlidone), etc. The microneedles can each have one or more recesses running a certain depth into the outer wall to allow for flow of the substance to be delivered down the microneedle and into the dermis; these recesses can be in a plurality of shapes, including but not limited to: straight line, cross shape (+), flat shape (−), or screw thread shape going clockwise or counterclockwise. The array can be in any shape or combination of shapes, continuous, or discontinuous. The list of possible shapes includes, but is not limited to, circles, triangles, rectangles, squares, rhomboids, trapezoids, and any other regular or irregular polygons. The array can be attached to a reservoir to hold the substances to be delivered, and this reservoir will be any volume (0.25 mL to 5 mL), shape, color, or material (glass, metal, alloy, or polymer), as determined necessary. This reservoir can itself be attached to or contain a means to encourage flow of the drug solutions contained in the reservoir into the skin. Two non-limiting examples of such means are 1) a plate and spring that allows the contained solutions to flow only when the device is tapped into the skin, and 2) a syringe that contains the drug solutions to be delivered and includes a plunger that can be depressed to mechanically drive the solution into the skin.

The delivered substances may be of varying viscosities and concentration, from 0.01% to 100%, and can be administered via the microneedle array either independently or in conjunction with the aforementioned compositions.

The reservoir can itself be attached to or contain a means to encourage flow of the drug solutions contained in the reservoir into the skin. Two non-limiting examples of such means are 1) a plate and spring that allows the contained solutions to flow only when the device is tapped into the skin, and 2) a syringe that contains the drug solutions to be delivered and includes a plunger that can be depressed to mechanically drive the solution into the skin.

A cadre of microneedles housed in a plastic or polymer composite head can be used to deliver treatment solutions, directly to the dermis, the second layer of skin or the topical layer of skin. The application of a mechanical load to the pin of the spring lock system enables the micro needles to puncture the epidermal barrier and deliver the desired substances directly to the dermis for faster, more efficient, and more effective absorption by the skin. The Spring Plate mechanism, housed in the plastic or polymer composite cartridge, is effectively the interface whereby the manual direct application mechanism calibrates the controlled delivery of the treatment solution into the skin.

In some embodiments, the treatment methods further comprise administering to the subject one or more additional therapies. In some embodiments, the additional therapy includes radiation, surgery, chemotherapy, simple excision, Mohs micrographic surgery, curettage and electrodesiccation, cryosurgery, photodynamic therapy, topical chemotherapy, topical immunotherapy (e.g., imiquimod), an intravenously administered therapeutic agent, and an orally administered therapeutic agent. In some embodiments, the additional therapeutic agent can be administered with a microneedle delivery device, alone or in combination with the antineoplastic agent.

As used herein, “treat” and all its forms and tenses (including, for example, treating, treated, and treatment) refers to therapeutic and prophylactic treatment. In certain aspects of the invention, those in need of treatment include those already with a pathological disease or condition of the invention, in which case treating refers to administering to a subject (including, for example, a human or other mammal in need of treatment) a therapeutically effective amount of a composition so that the subject has an improvement in a sign or symptom of a pathological condition of the invention. The improvement may be any observable or measurable improvement. Thus, one of skill in the art realizes that a treatment may improve the patient's condition, but may not be a complete cure of the disease or pathological condition. The terms “treating” or “treatment” or “to treat” or “alleviating” or “to alleviate” refer to both (1) therapeutic measures that cure, slow down, lessen symptoms of, and/or halt progression of a diagnosed pathologic condition or disorder and (2) prophylactic or preventative measures that prevent or slow the development of a targeted pathologic condition or disorder. Thus those in need of treatment include those already with the disorder; those prone to have the disorder; and those in whom the disorder is to be prevented. In the case of cancer or a tumor, a subject is successfully “treated” according to the methods of the present invention if the patient shows one or more of the following: a reduction in tumorigenicity, a reduction in the number or frequency of cancer stem cells, an increased immune response, an increased anti-tumor response, increased cytolytic activity of immune cells, increased killing of tumor cells, increased killing of tumor cells by immune cells, a reduction in the number of or complete absence of cancer cells; a reduction in the tumor size; inhibition of or an absence of cancer cell infiltration into peripheral organs including the spread of cancer cells into soft tissue and bone; inhibition of or an absence of tumor or cancer cell metastasis; inhibition or an absence of cancer growth; relief of one or more symptoms associated with the specific cancer; reduced morbidity and mortality; improvement in quality of life; or some combination of effects.

In accordance with the invention, a “therapeutically effective amount” or “effective amount” is administered to the subject. As used herein a “therapeutically effective amount” or “effective amount” is an amount sufficient to decrease, suppress, or ameliorate one or more symptoms associated with the disease or condition. The terms “effective amount” or “therapeutically effective amount” or “therapeutic effect” refer to an amount of an agent described herein, an antibody, a polypeptide, a polynucleotide, a small organic molecule, or other drug effective to “treat” a disease or disorder in a subject such as, a mammal. In the case of cancer or a tumor, the therapeutically effective amount of an agent (e.g., antibody or small molecule) has a therapeutic effect and as such can enhance or boost the immune response, enhance or boost the anti-tumor response, increase cytolytic activity of immune cells, increase killing of tumor cells, increase killing of tumor cells by immune cells, reduce the number of tumor cells; decrease tumorigenicity, tumorigenic frequency, or tumorigenic capacity; reduce the number or frequency of cancer stem cells; reduce the tumor size; reduce the cancer cell population; inhibit or stop cancer cell infiltration into peripheral organs including, for example, the spread of cancer into soft tissue and bone; inhibit and stop tumor or cancer cell metastasis; inhibit and stop tumor or cancer cell growth; relieve to some extent one or more of the symptoms associated with the cancer; reduce morbidity and mortality; improve quality of life; or a combination of such effects.

The term “pharmaceutically acceptable” refers to a substance approved or approvable by a regulatory agency of the Federal government or a state government or listed in the U.S. Pharmacopeia or other generally recognized pharmacopeia for use in animals, including humans.

The terms “pharmaceutically acceptable excipient, carrier, or adjuvant” or “acceptable pharmaceutical carrier” refer to an excipient, carrier, or adjuvant that can be administered to a subject, together with at least one agent of the present disclosure, and which does not destroy the pharmacological activity thereof and is non-toxic when administered in doses sufficient to deliver a therapeutic effect. In general, those of skill in the art and the U.S. FDA consider a pharmaceutically acceptable excipient, carrier, or adjuvant to be an inactive ingredient of any formulation.

The therapeutic compositions can be administered one time or more than one time, for example, more than once per day, daily, weekly, monthly, or annually. The duration of treatment is not particularly limiting. The duration of administration of the therapeutic composition can vary for each individual to be treated/administered depending on the individual cases and the diseases or conditions to be treated. In some embodiments, the therapeutic composition can be administered continuously for a period of several days, weeks, months, or years of treatment or can be intermittently administered where the individual is administered the therapeutic composition for a period of time, followed by a period of time where they are not treated, and then a period of time where treatment resumes as needed to treat the disease or condition. For example, in some embodiments, the individual to be treated is administered the therapeutic composition of the invention daily, every other day, every three days, every four days, 2 days per week 3 days per week, 4 days per week, 5 days per week or 7 days per week. In some embodiments, the individual is administered the therapeutic composition for 1 week, 2 weeks, 3 weeks, 4 weeks, 1 month, 2 months, 3 months, 4 months, 5 months, 6 months, 7 months, 8 months, 9 months, 10 months, 11 months, 1 year, or longer.

The term “subject” as used herein is not limiting and is used interchangeably with patient. In some embodiments, the term subject refers to animals, such as mammals and the like. For example, mammals contemplated include humans, primates, dogs, cats, sheep, cattle, goats, pigs, horses, chickens, mice, rats, rabbits, guinea pigs, and the like.

The terms “cancer” and “cancerous” as used herein refer to or describe the physiological condition in mammals in which a population of cells are characterized by unregulated cell growth. Examples of cancer include, but are not limited to, carcinoma, blastoma, sarcoma, and hematologic cancers such as lymphoma and leukemia.

The terms “tumor” and “neoplasm” as used herein refer to any mass of tissue that results from excessive cell growth or proliferation, either benign (non-cancerous) or malignant (cancerous) including pre-cancerous lesions.

The term “metastasis” as used herein refers to the process by which a cancer spreads or transfers from the site of origin to other regions of the body with the development of a similar cancerous lesion at a new location. Generally, a “metastatic” or “metastasizing” cell is one that loses adhesive contacts with neighboring cells and migrates via the bloodstream or lymph from the primary site of disease to secondary sites throughout the body.

The term “skin neoplasm,” as used herein, refers to a new and abnormal growth of tissue in the skin. In some embodiments, the neoplasm is malignant. Exemplary skin neoplasms include, but are not limited to: keratinocytic neoplasms, such as basal cell carcinoma, squamous cell carcinoma, Bowen's disease, bowenoid papulosis, Merkel cell carcinoma, actinic keratosis, and keratoacanthoma; melanocytic neoplasms, including all types of melanoma, such as superficial spreading melanoma, nodular melanoma, lentigo melanoma, acral-lentiginous melanoma, desmoplastic melanoma, nevoid melanoma, and amelanotic melanoma; appendageal neoplasms; soft tissue neoplasms; neural neoplasms; and cutaneous neoplasms. Skin neoplasms amenable to treatment using the methods of the present disclosure include benign, pre-malignant, malignant, and/or metastatic skin neoplasms.

In one embodiment, the neoplasm is basal cell carcinoma (BCC). BCC, a subtype of nonmelanoma skin cancer, is a malignancy arising from epidermal basal cells. BCC is a potentially fatal disease linked to sun exposure. The natural history of BCC is that of a slowly enlarging, locally invasive neoplasm. The degree of destruction and risk of recurrence vary with the size, duration and location of the tumor; the histologic subtype; the presence of recurrent disease; and various patient characteristics. Lesions located on the central face (e.g., the nose, the nasolabial fold, or the periorbital or perioral area), the ears, or the scalp are associated with a higher risk. Small nodular, pigmented, cystic, or superficial BCC respond well to treatments. Large nodular, micronodular, noduloulcerative, adenoid, infiltrative, and especially morpheaform BCCs tend to be more aggressive. Mortality rates due to BCC are low, but its increasing incidence and prolonged morbidity means the disease is costly to treat. Advanced lesions may ulcerate and extensive local invasion of bone or facial sinuses may occur. Early recognition and effective treatment are therefore important.

The methods of the invention can be combined with one or more additional treatments for BCC. Current treatments for BCC include electrodessication and curettage (ED&C), surgical excision, Mohs micrographic surgery (MMS), cryosurgery, radiation therapy, and treatment with 5-fluorouracil. Newer treatment modalities include photodynamic therapy and the topical application of a 5% imiquimod cream, which effectively resolves BCC lesions. The combination mode of therapy chosen can depends on tumor characteristics, age, medical status, preferences of the patient, and other factors. ED&C is the method commonly employed for low-risk tumors (e.g., a small primary tumor of a less aggressive subtype in a favorable location). Surgical excision, which offers the advantage of histologic control, is often selected for more aggressive tumors or those in high-risk locations, or, in many instances, for esthetic reasons. Cryosurgery using liquid nitrogen may be used in certain low-risk tumors. Radiation therapy, while not employed as often as surgical modalities, offers an excellent chance for cure in many cases of BCC. It is useful in patients not considered surgical candidates and as a surgical adjunct in high-risk tumors. MMS is a specialized type of surgical excision that permits the ultimate in histologic control and preservation of uninvolved tissue. It is preferred for recurrent lesions or lesions that are in a high-risk location or are large and ill defined, and where maximal tissue conservation is critical (e.g., the eyelids). Photodynamic therapy, which employs selective activation of a photoactive drug by visible light, may be useful in patients with numerous tumors. Lasers can also be used for the treatment of skin cancer. For reviews of BCC treatment modalities see, for example, Stockfleth and Sterry (Recent Results Cancer Res (2002) 160:259-68) and Kuijpers et al. (Am J Clin Dermatol 2002; 3(4):247-59).

In one embodiment, the neoplasm to be treated is squamous cell carcinoma (SCC). SCC, a subtype of nonmelanoma skin cancer, is the most common tumor arising in sun-exposed skin in older people. Implicated as predisposing factors, in addition to sunlight, are industrial carcinogens (tars and oils), chronic ulcers, old burn scars, ingestion of arsenicals, ionizing radiation, and (in the oral cavity) tobacco and betel nut chewing. Primary cutaneous SCC is a malignant neoplasm of keratinizing epidermal cells. Unlike BCC, which has very low metastatic potential, SCC can metastasize and grow rapidly. The clinical features of SCC can vary widely. Commonly, SCC first appears as an ulcerated nodule or a superficial erosion on the skin or lower lip. The margins of the tumor may be ill defined, and fixation to underlying structures may occur.

In some embodiments, the methods of the invention are combined with one or more additional treatments for SCC. Surgical excision, MMS, and radiation are standard methods of treatment of SCC. Cryosurgery and ED&C can be used, particularly for the treatment of small primary tumors. Metastases can be treated with lymph node dissection, irradiation, or both. Systemic chemotherapy combinations that include cisplatin may also be used for the treatment of metastatic SCC.

Before the development of overt malignancy of the epidermis, a series of progressively dysplastic changes occur. SCC has several premalignant forms (e.g., actinic keratosis, actinic cheilitis, and some cutaneous horns), and in situ forms (e.g., Bowen's disease) that are confined to the epidermis.

In one embodiment, the neoplasm to be treated is actinic keratosis (AK). Actinic keratoses are hyperkeratotic papules and plaques that occur on sun-exposed areas. Exposure to ionizing radiation, hydrocarbons, and arsenicals may induce similar lesions. Skin sites commonly affected can include the face, arms, scalp, and dorsum of the hands. Similar lesions may develop on the lips and are called actinic cheilitis. While the potential for malignant degeneration is low in individual lesions, the risk of SCC increases with larger numbers of AK lesions. AK lesions become malignant frequently enough to warrant local eradication of these potential precursor lesions.

In some embodiments, the methods of the invention are combined with one or more additional treatments for AK. In some embodiments, the combination includes curettage, cryotherapy, or topical application of chemotherapeutic agents.

In one embodiment, the neoplasm to be treated is Bowen's disease. Bowen's disease is a precancerous lesion, which presents as a scaling, erythematous plaque. It may develop into invasive SCC in up to 20% of cases. Thus treatment of the in situ lesions of Bowen's disease reduces the subsequent risk of invasive disease.

In some embodiments, the methods of the invention are combined with one or more additional treatments for Bowen's disease. In some embodiments, the combination includes surgical excision and direct closure. Alternative treatments can include cryotherapy, curettage and cautery, radiation, ultrasonic surgical aspiration (Otani et al., Plast Reconstr Surg (2001) 108(1):68-72), and photodynamic therapy (Wong et al., Dermatol Surg (2001) 27(5):452-6).

In one embodiment, the neoplasm to be treated is lentigo maligna. Lentigo maligna is a preinvasive form of melanoma induced by long-term cumulative ultraviolet injury. Lentigo maligna typically refers to lesions that are confined to the epidermis, whereas lentigo maligna melanoma typically refers to lesions that violate the dermis, thereby establishing metastatic potential. The most frequent findings suggesting early melanoma are changes in size or color of a new, pigmented lesion or an existing mole. Lentigo maligna most commonly affects the sun-exposed skin of the head and neck, with a predilection for the nose and cheek. Less common sites include the arm, leg, and trunk. The conjunctivae and oral mucosa may become involved when a cutaneous lentigo maligna spreads onto mucosal surfaces.

In some embodiments, the methods of the invention are combined with one or more additional treatments for lentigo maligna. In some embodiments, the combination includes radiotherapy, cryotherapy, chemotherapy, and/or surgical removal. Because the actual margins of the lesion usually extend beyond the clinically apparent margin, removal of the entire lesion may be difficult.

As used herein, “margin” and variations thereof refer to the tissue surrounding a neoplasm. More particularly, as used herein, “margins” refers to the edges, borders, or boundaries of a neoplasm. The margin is the region surrounding a neoplasm in which the normal, healthy tissue may have been altered by the presence of the neoplasm. For example, a tumor margin can include tumor cells that have grown beyond the visibly discernable edge of the tumor and can also include stromal regions that have been altered due to the presence of the tumor. In the case of ablation of a lesion, the margin includes tissues that usually appear to be normal to the naked eye that are removed along with the discernible lesion. The margin can generally extend from about 0.2 cm to about 3 cm from a primary lesion, but can be greater depending upon the size of the primary lesion.

As used herein, a “treatment area” is an area of the skin to which an antineoplastic agent is administered using a microneedle delivery device. In some embodiments, a treatment area may be defined by the presence of one or more clinically visible lesions. In such cases, the treatment area may include the clinically visible lesions, including the margins, as well as tissue located between clinically visible lesions. In other cases, a treatment area may be any area to which an antineoplastic agent is administered that lacks clinically visible lesions. For example, a treatment area may be selected because the area is at risk for developing certain types of skin lesions, e.g., areas of the face, scalp, neck, and hands may be at particular risk for developing actinic keratoses, basal cell carcinoma, melanoma, etc. Antineoplastic agents can be administered to such areas periodically as a treatment or to prevent the occurrence or progression of a neoplasm.

The antineoplastic agent that can be administered is not particularly limiting, and can include any of the therapeutic agents described herein, including those that are described for use in combination with the antineoplastic agents herein. Such agents can be administered alone or in combination with other therapeutic agents, including other antineoplastic agents.

In some embodiments, the antineoplastic agent that can be used in methods of the present disclosure include, but are not limited to, e.g., alkylating agents and platinum compounds (e.g., cisplatin, carboplatin, oxaliplatin, mechlorethamine, cyclophosphamide, chlorambucil, ifosfamide), anti-metabolic agents (e.g., purine and pyrimidine analogues, antifolates), anthracyclines (doxorubicin, daunorubicin, valrubicin, idarubicin, epirubicin), cytotoxic antibiotics (actinomycin, bleomycin, plicamycin, mitomycin), monoclonal antibodies (e.g., Alemtuzumab, Bevacizumab, Cetuximab, Gemtuzumab, Ibritumomab, Panitumumab, Rituximab, Tositumomab, and Trastuzumab), cancer antigens, checkpoint inhibitors, immune cells (activated immune cells, activated lymphocytes, engineered cells), kinase inhibitors (e.g., imatinib, erlotinib, gefitinib), plant alkaloids and terpenoids, topoisomerase inhibitors (e.g., camptothecins, irinotecan, topotecan, amsacrine, etoposide, etoposide phosphate, teniposide), vinca alkaloids (e.g., vincristine, vinblastine, vinorelbine, vindesine), taxanes (e.g., paclitaxel, taxol, docetaxel), podophyllotoxins, epipodophyllotoxins, and the like.

In some embodiments, the antineoplastic agent is selected from imiquimod, fluorouracil, vismodegib, 5-FU, sonidegib and combinations thereof. In some embodiments, these agents can be used to treat BCC.

In some embodiments, the antineoplastic agent is cemiplimab-rwlc. In some embodiments, cemiplimab-rwlc can be used to treat cutaneous squamous cell carcinoma.

In some embodiments, the antineoplastic agent is selected from aldesleukin, cobimetinib, dabrafenib, dacarbazine, IL-2, talimogene laherparepvec, interferon alfa-2b, ipilimumab, pembrolizumab, trametinib, nivolumab, nivolumab, peg-interferon Alfa-2b, pembrolizumab, aldesleukin, dabrafenib, trametinib, vemurafenib and combinations thereof. In some embodiments, these agents can be used to treat melanoma.

In some embodiments, the antineoplastic agent is selected from avelumab, pembrolizumab and a combination thereof. In some embodiments, these agents can be used to treat Merkel cell carcinoma.

In some embodiments, the antineoplastic agent is selected from afatinib/afatinib dimaleate, Tyrosine kinase inhibitor and combinations thereof. In some embodiments, these agents can be used to treat squamous cell carcinoma of head and neck, recurrent and/or metastatic disease, second-line as monotherapy after failure of platinum-based therapy.

In some embodiments, the antineoplastic agent is selected from atezolizumab, monoclonal antibodies, cobimetinib, vemurafenib and combinations thereof. In some embodiments, these agents can be used to treat malignant melanoma, unresectable or metastatic, BRAF V600 mutation positive.

In some embodiments, the antineoplastic agent is selected from avelumab, monoclonal antibodies and combinations thereof. In some embodiments, these agents can be used to treat Merkel cell carcinoma.

In some embodiments, the antineoplastic agent is selected from binimetinib, encorafenib and combinations thereof. In some embodiments, these agents can be used to treat malignant melanoma, unresectable or metastatic, with a BRAF V600E or V600K mutation.

In some embodiments, the antineoplastic agent is selected from cemiplimab-rwlc, monoclonal antibodies and combinations thereof. In some embodiments, these agents can be used to treat squamous cell carcinoma of skin, metastatic or locally advanced disease, in subjects who are not candidates for curative surgery or curative radiation.

In some embodiments, the antineoplastic agent is selected from cobimetinib, vemurafenib and combinations thereof. In some embodiments, these agents can be used to treat malignant melanoma, unresectable or metastatic with a BRAF V600E or V600K mutation.

In some embodiments, the antineoplastic agent is hydroxyurea. In some embodiments, these agents can be used to treat squamous cell carcinoma of head and neck, locally advanced disease, in combination with chemoradiation therapy.

In some embodiments, the antineoplastic agent is interferon gamma. In some embodiments, these agents can be used to treat atopic dermatitis.

In some embodiments, the antineoplastic agent is selected from lomustine, alkylating agent and combinations thereof. In some embodiments, these agents can be used to treat malignant melanoma.

In some embodiments, the antineoplastic agent is selected from olaratumab, monoclonal antibodies, doxorubicin and combinations thereof. In some embodiments, these agents can be used to treat soft tissue sarcoma, histologic subtype appropriate for an anthracycline-containing regimen which is not amenable to curative treatment with radiotherapy or surgery.

In some embodiments, the antineoplastic agent is pazopanib/pazopanib HCl. In some embodiments, these agents can be used to treat advanced soft tissue sarcoma.

In some embodiments, the antineoplastic agent is selected from tasonermin, TNF inhibitor and combinations thereof. In some embodiments, these agents can be used to treat soft tissue sarcoma.

In some embodiments, the antineoplastic agent is trametinib. In some embodiments, these agents can be used to treat malignant melanoma.

In some embodiments, the antineoplastic agent is selected from vemurafenib, BRAF inhibitor and combinations thereof. In some embodiments, these agents can be used to treat malignant melanoma. In some embodiments, the antineoplastic agent for use in the invention is selected from LDE225B, vismodegib, RIVEDGE, PD-L1, Patidegib, Nivolumab, Nivolumab+Ipilimumab, Imiquimod, Metvix PDT, Diclofenac, Diclofenac+Calcitriol, Calcitriol, Methylaminolevulinate, Fractionated 5-aminolevulinic acid hydrochloride, PEP005, SUBA-Itraconazole, methyl-aminolevulinatem, Remetinostat, verteporfin PDT, Sonidegib, Itraconazole, vismodegib, Picato, Resiquimod, API 31510, Aminolevulinic acid, arsenic trioxide, Patidegib, REGN2810, Buparlisib, Oshadi D & Oshadi R, Celecoxib, Tazarotene, Sinecatechins 10%, aminolevulinic acid hydrochloride, Vismodegib, FOLFOX, FOLFIRI, Bevacizumab, Tazarotene, Hexylaminolevulinate, Aminolevulinic Acid, Nano Emulsion, Methylaminolevulinate, liposomal T4N5 lotion, Carboplatin, Cyclophosphamide, Etoposide, Methotrexate, Vincristine Sulfate, Acetylcysteine, Bevacizumab, Eflornithine, Celecoxib, erlotinib hydrochloride, Poly-ICLC, aminolevulinic acid, eflornithine, triamcinolone, 18F-fludeoxyglucose (18F-FDG), 18F-FPPRGD2, Fluconazole, cevimeline hydrochloride, megestrol acetate, Amifostine, Carboplatin, Etoposide, Ifosfamide, Hypericum perforatum, Docetaxel, Nicotinamide, doxepin hydrochloride, capecitabine, oxaliplatin, DetoxPC, Capecitabine, Carboplatin, epirubicin hydrochloride, cisplatin, paclitaxel, Tretinoin, doxorubicin hydrochloride, gemcitabine hydrochloride, indinavir sulfate, ritonavir, 5-fluorouracil, and combinations thereof. In some embodiments, the antineoplastic agents above can be used to treat basal cell carcinoma. In some embodiments, the antineoplastic agents above can be used to treat actinic keratosis.

In some embodiments, the antineoplastic agent for use in the invention is selected from Imiquimod, VDA-1102, Fluorouracil, Cetaphil, SOR007 (Uncoated Nanoparticulate Paclitaxel), Aminolevulinic Acid, PEP005 (ingenol mebutate), ingenol disoxate, Ingenol Mebutate, Aminolevulinic Acid (ALA), Biafine, Polysporin, 5-FU, A-101 Solution (High-Concentration Hydrogen Peroxide), lidamycin phosphate and benzoyl peroxide 1.2%/3.75% combination, Eflornithine, Triamcinolone, Polyphenon E, BLU-U, ACT01, Levulan®, Kerastick®, perillyl alcohol, aminolevulinic acid, liposomal T4N5, Aminolevulinic Acid, GDC 695, Diclofenac Sodium, KX2-391, ingenol disoxate, ingenol disoxate, Celecoxib, Bupivacaine+Clonidine, Secukinumab, Pimecrolimus and combinations thereof. In some embodiments, the antineoplastic agents above can be used to treat basal cell carcinoma. In some embodiments, the antineoplastic agents above can be used to treat actinic keratosis.

In some embodiments, the antineoplastic agent useful in the invention comprises a nucleic acid molecule. In some embodiments, the nucleic acid molecule is capable of modulating the expression of one or more polypeptides in the cancer cell, such as one or more oncogenic polypeptides.

In some embodiments, the polypeptides are selected from RAS (H-,K-,N-), BRAF Cyclin D-1, CDK4, c-MYC, HDM-2, and MGSA/GRO. In some embodiments, expression of one or more of RAS (H-,K-,N-), BRAF, Cyclin D-1, CDK4, c-MYC, HDM-2, and MGSA/GRO can be modulated to treat melanoma.

In some embodiments, the polypeptides are selected from EGFR, CTNNB1, STK11, CDKN2A, HGF, MET, JUN and PAK-2. In some embodiments, expression of one or more of EGFR, CTNNB1, STK11, CDKN2A, HGF, MET, JUN and PAK-2 can be modulated to treat squamous cell carcinoma.

In some embodiments, the polypeptides are selected from GLI1, GLI2, Cyclin D, Cyclin E, and MYC. In some embodiments, expression of one or more of GLI1, GLI2, Cyclin D, Cyclin E, and MYC can be modulated to treat basal cell carcinoma.

In some embodiments, the polypeptides are selected from TP53, RAS and C-MYC. In some embodiments, expression of one or more of TP53, RAS and C-MYC can be modulated to treat actinic keratosis.

In some embodiments, the nucleic acid molecule comprises a nucleotide sequence that binds to at least a portion of a nucleotide sequence encoding a polypeptide of the cell. The nucleic acid molecule can be of any length, so long as at least part of the molecule hybridizes sufficiently and specifically to the nucleic acid target of interest. The nucleic acid molecule can bind to any region of the mRNA or DNA.

In some embodiments, a region of the nucleic acid molecule is at least 70%, 75%, 80%, 85%, 90%, 91%, 92%, 93%, 94%, 95%, 96%, 97%, 98% or 99% complementary to at least a portion of a nucleotide sequence of interest.

In some embodiments, the composition can comprise a DNA molecule, such as an antisense DNA molecule. In some embodiments, the composition can comprise an RNA molecule, such as an anti-sense RNA molecule, a small interfering RNA (siRNA) molecule, or small hairpin RNA (shRNA) molecule, which may or may not be comprised on a vector, including a viral vector (such as an adeno-associated viral vector, an adenoviral vector, a retroviral vector, or a lentiviral vector) or a non-viral vector. In some embodiments, the expression of the DNA or RNA molecule may be regulated by a regulatory region present in the cancer cells.

In some embodiments, the agent can be an RNA interference molecule; the RNA interference molecule may be a shRNA, siRNA, miRNA, or guide RNA to CRISPR/CAS9 CRISPRi, etc. Combinations of shRNAs can also be used in accordance with the present invention.

A target sequence on a target mRNA can be selected from a given cDNA sequence, in some embodiments, beginning 50 to 100 nt downstream (i.e., in the 3′ direction) from the start codon. In some embodiments, the target sequence can, however, be located in the 5′ or 3′ untranslated regions, or in the region nearby the start codon.

In some embodiments, the composition comprises an anti-sense DNA. Anti-sense DNA binds with mRNA and prevents translation of the mRNA. The anti-sense DNA can be complementary to a portion of the mRNA. In some embodiments, the anti-sense DNA is complementary to the entire reading frame. In some embodiments, the antisense DNA is at least 15 nucleotides, at least 20 nucleotides, at least 25 nucleotides, at least 30 nucleotides, at least 35 nucleotides, at least 40 nucleotides, at least 50 nucleotides, at least 75 nucleotides, at least 100 nucleotides, at least 150 nucleotides, at least 200 nucleotides, at least 300 nucleotides, at least 400 nucleotides, at least 500 nucleotides, at least 600 nucleotides, at least 700 nucleotides, at least 800 nucleotides, at least 900 nucleotides, at least 1000 nucleotides, at least 1500 nucleotides, at least 2000 nucleotides, at least 2500 nucleotides, at least 3000 nucleotides, at least 3500 nucleotides, at least 4000 nucleotides, or at least 4500 nucleotides.

In some embodiments, the composition comprises an anti-sense RNA. Anti-sense RNA binds with mRNA and prevents translation of the mRNA. The anti-sense RNA can be complementary to a portion of the mRNA. In some embodiments, the anti-sense RNA is complementary to the entire reading frame. In some embodiments, the antisense RNA is at least 15 nucleotides, at least 20 nucleotides, at least 25 nucleotides, at least 30 nucleotides, at least 35 nucleotides, at least 40 nucleotides, at least 50 nucleotides, at least 75 nucleotides, at least 100 nucleotides, at least 150 nucleotides, at least 200 nucleotides, at least 300 nucleotides, at least 400 nucleotides, at least 500 nucleotides, at least 600 nucleotides, at least 700 nucleotides, at least 800 nucleotides, at least 900 nucleotides, at least 1000 nucleotides, at least 1500 nucleotides, at least 2000 nucleotides, at least 2500 nucleotides, at least 3000 nucleotides, at least 3500 nucleotides, at least 4000 nucleotides, or at least 4500 nucleotides.

In some embodiments, the composition is an siRNA. SiRNAs are small single or dsRNAs that do not significantly induce the antiviral response common among vertebrate cells but that do induce target mRNA degradation via the RNAi pathway. The term siRNA refers to RNA molecules that have either at least one double stranded region or at least one single stranded region and possess the ability to effect RNA interference (RNAi). It is specifically contemplated that siRNA can refer to RNA molecules that have at least one double stranded region and possess the ability to effect RNAi. The dsRNAs (siRNAs) may be generated by various methods including chemical synthesis, enzymatic synthesis of multiple templates, digestion of long dsRNAs by a nuclease with RNAse III domains, and the like. An “siRNA directed to” at least a particular region means that a particular siRNA includes sequences that result in the reduction or elimination of expression of the target gene, i.e., the siRNA is targeted to the region or gene.

The nucleotide sequence of the siRNA is defined by the nucleotide sequence of its target gene. The siRNA contains a nucleotide sequence that is essentially identical to at least a portion of the target gene. In some embodiments, the siRNA contains a nucleotide sequence that is completely identical to at least a portion of the gene. Of course, when comparing an RNA sequence to a DNA sequence, an “identical” RNA sequence will contain ribonucleotides where the DNA sequence contains deoxyribonucleotides, and further that the RNA sequence will typically contain a uracil at positions where the DNA sequence contains thymidine.

In some embodiments, a siRNA comprises a double stranded structure, the sequence of which is “substantially identical” to at least a portion of the target gene. “Identity,” as known in the art, is the relationship between two or more polynucleotide (or polypeptide) sequences, as determined by comparing the sequences. In the art, identity also means the degree of sequence relatedness between polynucleotide sequences, as determined by the match of the order of nucleotides or amino acids between such sequences. Unless otherwise stated, sequence identity/similarity values provided herein refer to the value obtained using the BLAST 2.0 suite of programs using default parameters (Altschul, et al., (1997) Nucleic Acids Res. 25:3389-402).

One of skill in the art will appreciate that two polynucleotides of different lengths may be compared over the entire length of the longer fragment. Alternatively, small regions may be compared. Normally sequences of the same length are compared for a final estimation of their utility in the practice of the present invention. In some embodiments, there is 100% sequence identity between the dsRNA for use as siRNA and at least 15 contiguous nucleotides of the target gene, although a dsRNA having 70%, 75%, 80%, 85%, 90%, or 95% or greater may also be used in the present invention. A siRNA that is essentially identical to a least a portion of the target gene may also be a dsRNA wherein one of the two complementary strands (or, in the case of a self-complementary RNA, one of the two self-complementary portions) is either identical to the sequence of that portion or the target gene or contains one or more insertions, deletions or single point mutations relative to the nucleotide sequence of that portion of the target gene. siRNA technology thus has the property of being able to tolerate sequence variations that might be expected to result from genetic mutation, strain polymorphism, or evolutionary divergence.

In some embodiments, the invention provides an siRNA that is capable of triggering RNA interference, a process by which a particular RNA sequence is destroyed (also referred to as gene silencing). In specific embodiments, siRNA are dsRNA molecules that are 100 bases or fewer in length (or have 100 base pairs or fewer in its complementarity region). In some embodiments, a dsRNA may be 15, 16, 17, 18, 19, 20, 21, 22, 23, 24, 25, 26, 27, 28, 29, 30, 40, 50, 60, 70, 80, 90, 100 110, 120, 130, 140, 150, 160, 170, 180, 190, 200, 225, 250, 275, 300, 325, 350, 375, 400, 450, 500, 550, 600, 650, 700, 750, 800, 850, 900, 950, or 1000 nucleotides or more in length. In certain embodiments, siRNA may be approximately 21 to 25 nucleotides in length. In some cases, it has a two nucleotide 3′ overhang and a 5′ phosphate. The particular RNA sequence is targeted as a result of the complementarity between the dsRNA and the particular RNA sequence. It will be understood that dsRNA or siRNA of the disclosure can effect at least a 20, 30, 40, 50, 60, 70, 80, 90 percent or more reduction of expression of a targeted RNA in target cell. dsRNA of the invention (the term “dsRNA” will be understood to include “siRNA” and/or “candidate siRNA”) is distinct and distinguishable from antisense and ribozyme molecules by virtue of the ability to trigger RNAi. Structurally, dsRNA molecules for RNAi differ from antisense and ribozyme molecules in that dsRNA has at least one region of complementarity within the RNA molecule. In some embodiments, the complementary (also referred to as “complementarity”) region comprises at least or at most 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, or 500 contiguous bases. In some embodiments, long dsRNA are employed in which “long” refers to dsRNA that are 1000 bases or longer (or 1000 base pairs or longer in complementarity region). The term “dsRNA” includes “long dsRNA”, “intermediate dsRNA” or “small dsRNA” (lengths of 2 to 100 bases or base pairs in complementarity region) unless otherwise indicated. In some embodiments, dsRNA can exclude the use of siRNA, long dsRNA, and/or “intermediate” dsRNA (lengths of 100 to 1000 bases or base pairs in complementarity region).

It is specifically contemplated that a dsRNA may be a molecule comprising two separate RNA strands in which one strand has at least one region complementary to a region on the other strand. Alternatively, a dsRNA includes a molecule that is single stranded yet has at least one complementarity region as described above (such as when a single strand with a hairpin loop is used as a dsRNA for RNAi). For convenience, lengths of dsRNA may be referred to in terms of bases, which simply refers to the length of a single strand or in terms of base pairs, which refers to the length of the complementarity region. It is specifically contemplated that embodiments discussed herein with respect to a dsRNA comprised of two strands are contemplated for use with respect to a dsRNA comprising a single strand, and vice versa. In a two-stranded dsRNA molecule, the strand that has a sequence that is complementary to the targeted mRNA is referred to as the “antisense strand” and the strand with a sequence identical to the targeted mRNA is referred to as the “sense strand.” Similarly, with a dsRNA comprising only a single strand, it is contemplated that the “antisense region” has the sequence complementary to the targeted mRNA, while the “sense region” has the sequence identical to the targeted mRNA. Furthermore, it will be understood that sense and antisense region, like sense and antisense strands, are complementary (i.e., can specifically hybridize) to each other.

Strands or regions that are complementary may or may not be 100% complementary (“completely or fully complementary”). It is contemplated that sequences that are “complementary” include sequences that are at least 50% complementary, and may be at least 50%, 60%, 70%, 80%, or 90% complementary. In some embodiments, siRNA generated from sequence based on one organism may be used in a different organism to achieve RNAi of the cognate target gene. In other words, siRNA generated from a dsRNA that corresponds to a human gene may be used in a mouse cell if there is the requisite complementarity, as described above. Ultimately, the requisite threshold level of complementarity to achieve RNAi is dictated by functional capability. It is specifically contemplated that there may be mismatches in the complementary strands or regions. Mismatches may number at most or at least 1, 2, 3, 4, 5, 6, 7, 8, 9, 10, 11, 12, 13, 14, 15, 16, 17, 18, 19, 20, 21, 22, 23, 24, 25 residues or more, depending on the length of the complementarity region.

In some embodiments, the single RNA strand or each of two complementary double strands of a dsRNA molecule may be of at least or at most the following lengths: 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, or more (including the full-length mRNA without the poly-A tail) bases or base pairs. If the dsRNA is composed of two separate strands, the two strands may be the same length or different lengths. If the dsRNA is a single strand, in addition to the complementarity region, the strand may have 1, 2, 3, 4, 5, 6, 7, 8, 9, 10, 11, 12, 13, 14, 15, 16, 17, 18, 19, 20, 21, 22, 23, 24, 25, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more bases on either or both ends (5′ and/or 3′) or as forming a hairpin loop between the complementarity regions.

In some embodiments, the strand or strands of dsRNA are 100 bases (or base pairs) or less. In specific embodiments the strand or strands of the dsRNA are less than 70 bases in length. With respect to those embodiments, the dsRNA strand or strands may be from 5-70, 10-65, 20-60, 30-55, 40-50 bases or base pairs in length. A dsRNA that has a complementarity region equal to or less than 30 base pairs (such as a single stranded hairpin RNA in which the stem or complementary portion is less than or equal to 30 base pairs) or one in which the strands are 30 bases or fewer in length is specifically contemplated, as such molecules evade a mammalian's cell antiviral response. Thus, a hairpin dsRNA (one strand) may be 70 or fewer bases in length with a complementary region of 30 base pairs or fewer. In some cases, a dsRNA may be processed in the cell into siRNA.

The siRNA of the invention can comprise partially purified RNA, substantially pure RNA, synthetic RNA, or recombinantly produced RNA, as well as altered RNA that differs from naturally-occurring RNA by the addition, deletion, substitution and/or alteration of one or more nucleotides. Such alterations can include addition of non-nucleotide material, such as to the end(s) of the siRNA or to one or more internal nucleotides of the siRNA, including modifications that make the siRNA resistant to nuclease digestion.

One or both strands of the siRNA of the disclosure can comprise a 3′ overhang. As used herein, a “3′ overhang” refers to at least one unpaired nucleotide extending from the 3′-end of a duplexed RNA strand.

Thus in some embodiments, the siRNA of the invention comprises at least one 3′ overhang of from 1 to about 6 nucleotides (which includes ribonucleotides or deoxynucleotides) in length, from 1 to about 5 nucleotides in length, from 1 to about 4 nucleotides in length, or from about 2 to about 4 nucleotides in length.

In some embodiments in which both strands of the siRNA molecule comprise a 3′ overhang, the length of the overhangs can be the same or different for each strand. In some embodiments, the 3′ overhang is present on both strands of the siRNA, and is 2 nucleotides in length. For example, each strand of the siRNA of the invention can comprise 3′ overhangs of dithymidylic acid (“TT”) or diuridylic acid (“UU”).

In order to enhance the stability of the siRNA, the 3′ overhangs can be also stabilized against degradation. In some embodiments, the overhangs are stabilized by including purine nucleotides, such as adenosine or guanosine nucleotides. In some embodiments, substitution of pyrimidine nucleotides by modified analogues, e.g., substitution of uridine nucleotides in the 3′ overhangs with 2′-deoxythymidine, is tolerated and does not affect the efficiency of RNAi degradation. In particular, the absence of a 2′ hydroxyl in the 2′-deoxythymidine can significantly enhance the nuclease resistance of the 3′ overhang in tissue culture medium.

In some embodiments, the siRNA of the disclosure can be targeted to any stretch of approximately 19-25 contiguous nucleotides in any of the target mRNA sequences (the “target sequence”). Techniques for selecting target sequences for siRNA are given, for example, in Tuschl T et al., “The siRNA User Guide,” revised Oct. 11, 2002, the entire disclosure of which is herein incorporated by reference. “The siRNA User Guide” is available on the world wide web at a website maintained by Dr. Thomas Tuschl, Department of Cellular Biochemistry, AG 105, Max-Planck-Institute for Biophysical Chemistry, 37077 Gottingen, Germany, and can be found by accessing the website of the Max Planck Institute and searching with the keyword “siRNA.” Thus, in some embodiments, the sense strand of the present siRNA comprises a nucleotide sequence identical to any contiguous stretch of about 19 to about 25 nucleotides in the target mRNA.

In some embodiments, the siRNA comprises a 21 nucleotide double stranded sequence. In some embodiments, the siRNA comprises a two-TT overhang (Yang et al., Nucleic Acid Research, 34(4), 1224-1236, 2006).

In some embodiments, the composition useful in the methods of the invention comprises an shRNA molecule that targets mRNA. shRNA is an artificial RNA molecule with a tight hairpin turn that can be used to silence target gene expression via RNA interference (RNAi). In certain cases, expression of shRNA in cells is achieved through delivery of non-viral vectors (such as plasmids or bacterial vectors) or through viral vectors. shRNA is useful because it has a relatively low rate of degradation and turnover.

In order to obtain long-term gene silencing, expression vectors that continually express siRNAs in stably transfected mammalian cells can be used (Brummelkamp et al., Science 296: 550-553, 2002; Lee et al., Nature Biotechnol. 20:500-505, 2002; Miyagishi, M, and Taira, K. Nature Biotechnol. 20:497-500, 2002; Paddison, et al., Genes & Dev. 16:948-958, 2002; Paul et al., Nature Biotechnol. 20:505-508, 2002; Sui, Proc. Natl. Acad. Sci. USA 99(6):5515-5520, et al., 2002; Yu et al., Proc. Natl. Acad. Sci. USA 99(9):6047-6052, 2002). Many of these plasmids have been engineered to express shRNAs lacking poly (A) tails. Transcription of shRNAs is initiated at a polymerase III (pol III) promoter and is believed to be terminated at position 2 of a 4-5-thymine transcription termination site. Upon expression, shRNAs are thought to fold into a stem-loop structure with 3′ UU-overhangs. Subsequently, the ends of these shRNAs are processed, converting the shRNAs into ^{˜}21 nt siRNA-like molecules. The siRNA-like molecules can, in turn, bring about gene-specific silencing in the transfected mammalian cells.

The length of the stem and loop of shRNAs can be varied. In some embodiments, stem lengths could range anywhere from 25 to 29 nucleotides and loop size could range between 4 to 23 nucleotides without affecting silencing activity. Moreover, presence of G-U mismatches between the two strands of the shRNA stem does not necessarily lead to a decrease in potency.

In some embodiments, the present invention is directed to methods of administering subjects with compositions comprising expression vectors and/or chemically synthesized shRNA molecules that target certain genes in the cancer cells. In some embodiments, the composition comprises a nucleotide sequence expressing a small hairpin RNA (shRNA) molecule. In some embodiments, the expression vector is a lentivirus expression vector.

In some embodiments, it is contemplated that nucleic acids or other active agents of the invention may be labeled. The label may be fluorescent, radioactive, enzymatic, or calorimetric. It is contemplated that a dsRNA may have one label attached to it or it may have more than one label attached to it. When more than one label is attached to a dsRNA, the labels may be the same or be different. If the labels are different, they may appear as different colors when visualized. The label may be on at least one end and/or it may be internal. Furthermore, there may be a label on each end of a single stranded molecule or on each end of a dsRNA made of two separate strands. The end may be the 3′ and/or the 5′ end of the nucleic acid. A label may be on the sense strand or the sense end of a single strand (end that is closer to sense region as opposed to antisense region), or it may be on the antisense strand or antisense end of a single strand (end that is closer to antisense region as opposed to sense region). In some cases, a strand is labeled on a particular nucleotide (G, A, U, or C). When two or more differentially colored labels are employed, fluorescent resonance energy transfer (FRET) techniques may be employed to characterize the dsRNA.

Labels contemplated for use in several embodiments are non-radioactive. In many embodiments of the invention, the labels are fluorescent, though they may be enzymatic, radioactive, or positron emitters. Fluorescent labels that may be used include, but are not limited to, BODIPY, Alexa Fluor, fluorescein, Oregon Green, tetramethylrhodamine, Texas Red, rhodamine, cyanine dye, or derivatives thereof. The labels may also more specifically be Alexa 350, Alexa 430, AMCA, BODIPY 630/650, BODIPY 650/665, BODIPY-FL, BODIPY-R6G, BODIPY-TMR, BODIPY-TRX, Cascade Blue, Cy3, Cy5, DAPI, 6-FAM, Killer Red, Fluorescein Isothiocyanate, HEX, 6-JOE, Oregon Green 488, Oregon Green 500, Oregon Green 514, Pacific Blue, REG, Rhodamine Green, Rhodamine Red, Renographin, ROX, SYPRO, TAMRA, TET, Tetramethylrhodamine, and/or Texas Red. A labeling reagent is a composition that comprises a label and that can be incubated with the nucleic acid to effect labeling of the nucleic acid under appropriate conditions. In some embodiments, the labeling reagent comprises an alkylating agent and a dye, such as a fluorescent dye. In some embodiments, a labeling reagent comprises an alkylating agent and a fluorescent dye such as Cy3, Cy5, or fluorescein (FAM). In still further embodiments, the labeling reagent is also incubated with a labeling buffer, which may be any buffer compatible with physiological function (i.e., buffers that is not toxic or harmful to a cell or cell component) (termed “physiological buffer”).

In some embodiments, the nucleic acids of the invention can be modified. In some embodiments, the nucleic acids can be modified to include a phosphorothioate (PS) backbone. The modification to the backbone can be throughout the molecule or at one or more defined sites. In some embodiments, the nucleic acids can be modified to encompass peptide nucleic acids (PNA). In some embodiments, the nucleic acids can be modified to encompass phosphorodiamidate morpholino oligomers (PMO).

In some embodiments, the nucleic acid molecules of the invention can include derivatives such as S-oligonucleotides (phosphorothioate derivatives or S-oligos). S-oligos (nucleoside phosphorothioates) are isoelectronic analogs of an oligonucleotide (0-oligo) in which a nonbridging oxygen atom of the phosphate group is replaced by a sulfur atom. The S-oligos of the present invention may be prepared by treatment of the corresponding 0-oligos with 3H-1,2-benzodithiol-3-one-1,1-dioxide which is a sulfur transfer reagent. See Iyer et al., J. Org. Chem. 55:4693-4698 (1990); and Iyer et al., J. Am. Chem. Soc. 112:1253-1254 (1990), the disclosures of which are fully incorporated by reference herein.

In some embodiments of the invention, a dsRNA has one or more non-natural nucleotides, such as a modified residue or a derivative or analog of a natural nucleotide. Any modified residue, derivative or analog may be used to the extent that it does not eliminate or substantially reduce (by at least 50%) RNAi activity of the dsRNA.

A person of ordinary skill in the art is well aware of achieving hybridization of complementary regions or molecules. Such methods typically involve heat and slow cooling of temperature during incubation, for example.

In some embodiments, the nucleic acid molecules of the present methods are encoded by expression vectors. The expression vectors may be obtained and introduced into a cell. Once introduced into the cell the expression vector is transcribed to produce various nucleic acids. Expression vectors include nucleic acids that provide for the transcription of a particular nucleic acid. Expression vectors include plasmid DNA, linear expression elements, circular expression elements, viral expression constructs (including adenoviral, adeno-associated viral, retroviral, lentiviral, and so forth), and the like, all of which are contemplated as being used in the compositions and methods of the present disclosure. In some embodiments one or at least 2, 3, 4, 5, 6, 7, 8, 9, 10 or more nucleic acid molecules binding to one or more target nucleic acids are encoded by a single expression construct. Expression of the nucleic acid molecules binding to target RNA may be independently controlled by at least 1, 2, 3, 4, 5, 6, 7, 8, 9, 10 or more regulatory elements. In certain embodiments, at least 1, 2, 3, 4, 5, 6, 7, 8, 9, 10 or more expression constructs can be introduced into a cell. Each expression construct can encode 1, 2, 3, 4, 5, 6, 7, 8, 9, 10 or more nucleic acid molecules binding to the target RNA. In some embodiments, nucleic acid molecules binding to the target RNA may be encoded as expression domains. Expression domains include a transcription control element, which may or may not be independent of other control or promoter elements; a nucleic acid; and optionally a transcriptional termination element.

In some embodiments, the invention provides nucleic acid molecules encoding dominant negative or tumor suppressor polypeptides. In some embodiments, the nucleic acid molecule is packaged in a viral vector. In some embodiments, the dominant negative or tumor suppressor polypeptide or a biologically active fragment or derivative thereof may be derived from genomic DNA, i.e., cloned directly from the genome of a particular organism. In some embodiments, however, the vector comprising the dominant negative or tumor suppressor polypeptide comprises complementary DNA (cDNA).

The nucleic acid molecules may be produced using recombinant DNA technology (e.g., polymerase chain reaction (PCR) amplification, cloning) or chemical synthesis. The nucleic acids include natural nucleic acid molecules and homologues thereof, including, but not limited to, natural allelic variants and modified nucleic acid molecules in which nucleotides have been inserted, deleted, substituted, and/or inverted in such a manner that such modifications provide the desired effect.

Any suitable viral vector can be used in the methods of the invention. For example, vectors derived from adenovirus (AV); adeno-associated virus (AAV; including AAV serotypes); retroviruses (e.g, lentiviruses (LV), Rhabdoviruses, murine leukemia virus); herpes virus, and the like. The tropism of the viral vectors can also be modified by pseudotyping the vectors with envelope proteins or other surface antigens from other viruses. For example, an AAV vector of the invention can be pseudotyped with surface proteins from vesicular stomatitis virus (VSV), rabies, Ebola, Mokola, and the like.

Selection of recombinant viral vectors suitable for use in the invention, are within the skill in the art. See, for example, Dornburg R (1995), Gene Therap. 2: 301-310; Eglitis M A (1988), Biotechniques 6: 608-614; Miller A D (1990), Hum Gene Therap. 1: 5-14; and Anderson W F (1998), Nature 392: 25-30, the entire disclosures of which are herein incorporated by reference.

The ability of a RNA to cause RNAi-mediated degradation of the target mRNA can be evaluated using standard techniques for measuring the levels of RNA or protein in cells. For example, siRNA of the invention can be delivered to cultured cells, and the levels of target mRNA can be measured by Northern blot or dot blotting techniques, or by quantitative RT-PCR. Alternatively, the levels of protein in the cultured cells can be measured by ELISA or Western blot. A suitable cell culture system for measuring the effect of the present siRNA on target mRNA or protein levels may be utilized. RNAi-mediated degradation of mRNA by an siRNA containing a given target sequence can also be evaluated with animal models, for example.

In some embodiments, the nucleic acids can be administered to the subject either as naked nucleic acid, in conjunction with a delivery reagent, or as a recombinant plasmid or viral vector that expresses the nucleic acids. Delivery of nucleic acids or vectors to an individual may occur by any suitable means, but in specific embodiments it occurs by one of the following: cyclodextrin delivery system; ionizable lipids; DPC conjugates; GalNAc-conjugates; self-assembly of oligonucleotide nanoparticles (DNA tetrahedra carrying multiple siRNAs); or polymeric nanoparticles made of low-molecular-weight polyamines and lipids (see Kanasty et al. Nature Materials 12, 967-977 (2013) for review of same).

Suitable delivery reagents for administration in conjunction with the present nucleic acids or vectors include at least the Mirus Transit TKO lipophilic reagent; lipofectin; lipofectamine; cellfectin; or polycations (e.g., polylysine), or liposomes. In specific embodiments, a particular delivery reagent comprises a liposome.

Liposomes can aid in the delivery of the present nucleic acids or vectors to a particular tissue, and can also increase the blood half-life of the nucleic acids. Liposomes suitable for use in the invention can be formed from standard vesicle-forming lipids, which generally include neutral or negatively charged phospholipids and a sterol, such as cholesterol. The selection of lipids is generally guided by consideration of factors such as the desired liposome size and half-life of the liposomes in the blood stream. A variety of methods are known for preparing liposomes, for example as described in Szoka et al. (1980), Ann. Rev. Biophys. Bioeng. 9: 467; and U.S. Pat. Nos. 4,235,871, 4,501,728, 4,837,028, and 5,019,369, the entire disclosures of which are herein incorporated by reference.

In certain aspects, the liposomes encapsulating the present nucleic acids comprise a ligand molecule that can target the liposome to a particular cell or tissue at or near the site of interest. Ligands that bind to receptors prevalent in the tissues to be targeted, such as monoclonal antibodies that bind to surface antigens, are contemplated. In particular cases, the liposomes are modified so as to avoid clearance by the mononuclear macrophage and reticuloendothelial systems, for example by having opsonization-inhibition moieties bound to the surface of the structure. In one embodiment, a liposome of the invention can comprise both opsonization-inhibition moieties and a ligand. Opsonization-inhibiting moieties for use in preparing the liposomes of the disclosure are typically large hydrophilic polymers that are bound to the liposome membrane. As used herein, an opsonization inhibiting moiety is “bound” to a liposome membrane when it is chemically or physically attached to the membrane, e.g., by the intercalation of a lipid-soluble anchor into the membrane itself, or by binding directly to active groups of membrane lipids. These opsonization-inhibiting hydrophilic polymers form a protective surface layer which significantly decreases the uptake of the liposomes by the macrophage-monocyte system (“MMS”) and reticuloendothelial system (“RES”); e.g., as described in U.S. Pat. No. 4,920,016, the entire disclosure of which is herein incorporated by reference. Liposomes modified with opsonization-inhibition moieties thus remain in the circulation much longer than unmodified liposomes. For this reason, such liposomes are sometimes called “stealth” liposomes.

Stealth liposomes are known to accumulate in tissues fed by porous or “leaky” microvasculature. Thus, target tissue characterized by such microvasculature defects, for example solid tumors, will efficiently accumulate these liposomes; see Gabizon, et al. (1988), P.N.A.S., USA, 18: 6949-53. In addition, the reduced uptake by the RES lowers the toxicity of stealth liposomes by preventing significant accumulation in the liver and spleen. Thus, liposomes of the invention that are modified with opsonization-inhibition moieties can deliver the present nucleic acids to tumor cells.

In some embodiments, opsonization inhibiting moieties suitable for modifying liposomes are water-soluble polymers with a number-average molecular weight from about 500 to about 40,000 Daltons, and in some embodiments. from about 2,000 to about 20,000 Daltons. Such polymers can include polyethylene glycol (PEG) or polypropylene glycol (PPG) derivatives; e.g., methoxy PEG or PPG, and PEG or PPG stearate; synthetic polymers such as polyacrylamide or poly N-vinyl pyrrolidone; linear, branched, or dendrimeric polyamidoamines; polyacrylic acids; polyalcohols, e.g., polyvinylalcohol and polyxylitol to which carboxylic or amino groups are chemically linked, as well as gangliosides, such as ganglioside GM1. Copolymers of PEG, methoxy PEG, or methoxy PPG, or derivatives thereof, are also suitable. In addition, the opsonization inhibiting polymer can be a block copolymer of PEG and either a polyamino acid, polysaccharide, polyamidoamine, polyethyleneamine, or polynucleotide. The opsonization inhibiting polymers can also be natural polysaccharides containing amino acids or carboxylic acids, e.g., galacturonic acid, glucuronic acid, mannuronic acid, hyaluronic acid, pectic acid, neuraminic acid, alginic acid, carrageenan; aminated polysaccharides or oligosaccharides (linear or branched); or carboxylated polysaccharides or oligosaccharides, e.g., reacted with derivatives of carbonic acids with resultant linking of carboxylic groups.

In some embodiments the opsonization-inhibiting moiety is a PEG, PPG, or derivatives thereof. Liposomes modified with PEG or PEG-derivatives are sometimes called “PEGylated liposomes.” The opsonization inhibiting moiety can be bound to the liposome membrane by any one of numerous well-known techniques. For example, an N-hydroxysuccinimide ester of PEG can be bound to a phosphatidyl-ethanolamine lipid-soluble anchor, and then bound to a membrane. Similarly, a dextran polymer can be derivatized with a stearylamine lipid-soluble anchor via reductive amination using Na(CN)BH₃ and a solvent mixture such as tetrahydrofuran and water in a 30:12 ratio at 60 degrees C.

Recombinant plasmids that express nucleic acids of the invention are discussed above. Such recombinant plasmids can also be administered directly or in conjunction with a suitable delivery reagent, including the Mirus Transit LT 1 lipophilic reagent; lipofectin; lipofectamine; cellfectin; polycations (e.g., polylysine) or liposomes.

In certain embodiments, the methods of the invention may include a diagnostic step. Individuals may be diagnosed as being in need of the methods herein using any convenient protocol suitable for use in diagnosing the presence of a skin neoplasm, such as visual diagnosis, biopsy, dermatoscopy, etc. In addition, individuals may be known to be in need of the methods described herein, e.g., they are suffering from a skin neoplasm. Methods of the present disclosure may further include assessing the efficacy of the treatment protocol, which may be performed using any convenient protocol, e.g., by monitoring the rate of regression and/or progression of the skin neoplasm conditions (such as by using the diagnosis protocols, e.g., as described above).

In some embodiments, the methods herein further comprise one or more additional treatments for cancer. Combination therapy with two or more therapeutic agents often uses agents that work by different mechanisms of action, although this is not required. Combination therapy using agents with different mechanisms of action may result in additive or synergetic effects. Combination therapy may allow for a lower dose of each agent than is used in monotherapy, thereby reducing toxic side effects and/or increasing the therapeutic index of the agent(s). Combination therapy may decrease the likelihood that resistant cancer cells will develop. In some embodiments, combination therapy comprises a therapeutic agent that affects the immune response (e.g., enhances or activates the response) and a therapeutic agent that affects (e.g., inhibits or kills) the tumor/cancer cells.

In some embodiments, the combination of an agent described herein and at least one additional therapeutic agent results in additive or synergistic results. In some embodiments, the combination therapy results in an increase in the therapeutic index of the agent. In some embodiments, the combination therapy results in an increase in the therapeutic index of the additional therapeutic agent(s). In some embodiments, the combination therapy results in a decrease in the toxicity and/or side effects of the agent. In some embodiments, the combination therapy results in a decrease in the toxicity and/or side effects of the additional therapeutic agent(s).

In certain embodiments, the method or treatment further comprises administering at least one additional therapeutic agent. An additional therapeutic agent can be administered prior to, concurrently with, and/or subsequently to, administration of the antineoplastic agent. In some embodiments, the at least one additional therapeutic agent comprises 1, 2, 3, or more additional therapeutic agents.

Therapeutic agents that may be administered in combination with the agents described herein include one or more chemotherapeutic agents. Thus, in some embodiments, the method or treatment involves the administration of an antineoplastic agent of the present invention in combination with a chemotherapeutic agent or in combination with a cocktail of chemotherapeutic agents. Treatment with an agent can occur prior to, concurrently with, or subsequent to administration of chemotherapies. Combined administration can include co-administration, either in a single pharmaceutical formulation or using separate formulations, or consecutive administration in either order but generally within a time period such that all active agents can exert their biological activities simultaneously. Preparation and dosing schedules for such chemotherapeutic agents can be used according to manufacturers' instructions or as determined empirically by the skilled practitioner. Preparation and dosing schedules for such chemotherapy are also described in The Chemotherapy Source Book. 4^{th} Edition, 2008, M. C. Perry, Editor, Lippincott, Williams & Wilkins, Philadelphia, Pa.

Useful classes of therapeutic agents include, for example, anti-tubulin agents, auristatins, DNA minor groove binders, DNA replication inhibitors, alkylating agents (e.g., platinum complexes such as cisplatin, mono(platinum), bis(platinum) and tri-nuclear platinum complexes and carboplatin), anthracyclines, antibiotics, anti-folates, antimetabolites, chemotherapy sensitizers, duocarmycins, etoposides, fluorinated pyrimidines, ionophores, lexitropsins, nitrosoureas, platinols, purine antimetabolites, puromycins, radiation sensitizers, steroids, taxanes, topoisomerase inhibitors, vinca alkaloids, or the like. In certain embodiments, the second therapeutic agent is an alkylating agent, an antimetabolite, an antimitotic, a topoisomerase inhibitor, or an angiogenesis inhibitor.

Chemotherapeutic agents useful in the instant invention include, but are not limited to, alkylating agents such as thiotepa and cyclosphosphamide (CYTOXAN); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamime; nitrogen mustards such as chlorambucil, chlomaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, caminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytosine arabinoside, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenishers such as folinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PS K; razoxane; sizofuran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabino side (Ara-C); taxoids, e.g. paclitaxel (TAXOL) and docetaxel (TAXOTERE); chlorambucil; gemcitabine; 6-thioguanine; mercaptopurine; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; ibandronate; CPT11; topoisomerase inhibitor RFS 2000; difluoromethylornithine (DMFO); retinoic acid; esperamicins; capecitabine (XELODA); and pharmaceutically acceptable salts, acids or derivatives of any of the above. Chemotherapeutic agents also include anti-hormonal agents that act to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY117018, onapristone, and toremifene (FARESTON); and anti-androgens such as flutamide, nilutamide, bicalutamide, leuprolide, and goserelin; and pharmaceutically acceptable salts, acids or derivatives of any of the above. In certain embodiments, the additional therapeutic agent is cisplatin. In certain embodiments, the additional therapeutic agent is carboplatin. In certain embodiments, a combination of cisplatin and paclitaxel is administered, optionally in combination with one or more additional antineoplastic agents described herein.

In certain embodiments, the chemotherapeutic agent is a topoisomerase inhibitor. Topoisomerase inhibitors are chemotherapy agents that interfere with the action of a topoisomerase enzyme (e.g., topoisomerase I or II). Topoisomerase inhibitors include, but are not limited to, doxorubicin HCl, daunorubicin citrate, mitoxantrone HCl, actinomycin D, etoposide, topotecan HCl, teniposide (VM-26), and irinotecan, as well as pharmaceutically acceptable salts, acids, or derivatives of any of these. In some embodiments, the additional therapeutic agent is irinotecan.

In certain embodiments, the chemotherapeutic agent is an anti-metabolite. An anti-metabolite is a chemical with a structure that is similar to a metabolite required for normal biochemical reactions, yet different enough to interfere with one or more normal functions of cells, such as cell division. Anti-metabolites include, but are not limited to, gemcitabine, fluorouracil, capecitabine, methotrexate sodium, ralitrexed, pemetrexed, tegafur, cytosine arabinoside, thioguanine, 5-azacytidine, 6-mercaptopurine, azathioprine, 6-thioguanine, pentostatin, fludarabine phosphate, and cladribine, as well as pharmaceutically acceptable salts, acids, or derivatives of any of these. In certain embodiments, the additional therapeutic agent is gemcitabine.

In certain embodiments, the chemotherapeutic agent is an antimitotic agent, including, but not limited to, agents that bind tubulin. In some embodiments, the agent is a taxane. In certain embodiments, the agent is paclitaxel or docetaxel, or a pharmaceutically acceptable salt, acid, or derivative of paclitaxel or docetaxel. In certain embodiments, the agent is paclitaxel (TAXOL), docetaxel (TAXOTERE), albumin-bound paclitaxel (ABRAXANE), DHA-paclitaxel, or PG-paclitaxel. In certain alternative embodiments, the antimitotic agent comprises a vinca alkaloid, such as vincristine, vinblastine, vinorelbine, or vindesine, or pharmaceutically acceptable salts, acids, or derivatives thereof. In some embodiments, the antimitotic agent is an inhibitor of kinesin Eg5 or an inhibitor of a mitotic kinase such as Aurora A or Plk1. In certain embodiments, the additional therapeutic agent is paclitaxel. In certain embodiments, the additional therapeutic agent is albumin-bound paclitaxel (ABRAXANE).

In some embodiments, an additional therapeutic agent comprises an agent such as a small molecule. For example, treatment can involve the combined administration of an agent of the present invention with a small molecule that acts as an inhibitor against tumor-associated antigens including, but not limited to, EGFR, HER2 (ErbB2), and/or VEGF. In some embodiments, an agent of the present invention is administered in combination with a protein kinase inhibitor selected from the group consisting of: gefitinib (IRESSA), erlotinib (TARCEVA), sunitinib (SUTENT), lapatanib, vandetanib (ZACTIMA), AEE788, CI-1033, cediranib (RECENTIN), sorafenib (NEXAVAR), and pazopanib (GW786034B). In some embodiments, an additional therapeutic agent comprises an mTOR inhibitor.

In certain embodiments, the additional therapeutic agent is an agent that inhibits a cancer stem cell pathway. In some embodiments, the additional therapeutic agent is an inhibitor of the Notch pathway. In some embodiments, the additional therapeutic agent is an inhibitor of the Wnt pathway. In some embodiments, the additional therapeutic agent is an inhibitor of the BMP pathway. In some embodiments, the additional therapeutic agent is an inhibitor of the Hippo pathway. In some embodiments, the additional therapeutic agent is an inhibitor of the RSPO/LGR pathway. In some embodiments, the additional therapeutic agent is an inhibitor of the mTOR/AKR pathway.

In some embodiments, an additional therapeutic agent comprises a biological molecule, such as an antibody. For example, treatment can involve the combined administration of an agent of the present invention with antibodies against tumor-associated antigens including, but not limited to, antibodies that bind EGFR, HER2/ErbB2, and/or VEGF. In certain embodiments, the additional therapeutic agent is an antibody specific for a cancer stem cell marker. In some embodiments, the additional therapeutic agent is an antibody that binds a component of the Notch pathway. In some embodiments, the additional therapeutic agent is an antibody that binds a component of the Wnt pathway. In certain embodiments, the additional therapeutic agent is an antibody that inhibits a cancer stem cell pathway. In some embodiments, the additional therapeutic agent is an inhibitor of the Notch pathway. In some embodiments, the additional therapeutic agent is an inhibitor of the Wnt pathway. In some embodiments, the additional therapeutic agent is an inhibitor of the BMP pathway. In some embodiments, the additional therapeutic agent is an antibody that inhibits β-catenin signaling. In certain embodiments, the additional therapeutic agent is an antibody that is an angiogenesis inhibitor (e.g., an anti-VEGF or VEGF receptor antibody). In certain embodiments, the additional therapeutic agent is bevacizumab (AVASTIN), ramucirumab, trastuzumab (HERCEPTIN), pertuzumab (OMNITARG), panitumumab (VECTIBIX), nimotuzumab, zalutumumab, or cetuximab (ERBITUX).

In certain embodiments, the antineoplastic agent described herein is administered in combination with at least one immunotherapeutic agent. In some embodiments, the immunotherapeutic agent is an immune response stimulating agent. In some embodiments, the immunotherapeutic agent (e.g., immune response stimulating agent) includes, but is not limited to, a colony stimulating factor (e.g., granulocyte-macrophage colony stimulating factor (GM-CSF), macrophage colony stimulating factor (M-CSF), granulocyte colony stimulating factor (G-CSF), stem cell factor (SCF)), an interleukin (e.g., IL-1, IL2, IL-3, IL-7, I1-12, IL-15, IL-18), an antibody that blocks immunosuppressive functions (e.g., an anti-CTLA4 antibody, anti-CD28 antibody, anti-CD3 antibody, anti-PD-1 antibody, anti-PD-L1 antibody), an antibody that enhances immune cell functions (e.g., an anti-GITR antibody or an anti-OX-40 antibody), a toll-like receptor (e.g., TLR4, TLR7, TLR9), a soluble ligand (e.g., GITRL or OX-40L), or a member of the B7 family (e.g., CD80, CD86). An immunotherapeutic agent (e.g., an immune response stimulating agent) can be administered prior to, concurrently with, and/or subsequently to, administration of the antineoplastic agent described herein. Pharmaceutical compositions comprising an antineoplastic agent described herein and an immunotherapeutic agent (e.g., an immune response stimulating agent(s)) are also provided. In some embodiments, the immunotherapeutic agent comprises 1, 2, 3, or more immunotherapeutic agents. In some embodiments, the immune response stimulating agent comprises 1, 2, 3, or more immune response stimulating agents.

In some embodiments, the additional therapeutic agent is an antibody that is an immune checkpoint inhibitor. In some embodiments, the immune checkpoint inhibitor is an anti-PD-1 antibody, an anti-PD-L1 antibody, an anti-CTLA4 antibody, an anti-CD28 antibody, an anti-LAG3 antibody, an anti-TIM3 antibody, an anti-GITR antibody, or an anti-OX-40 antibody. In some embodiments, the immune checkpoint inhibitor is an anti-4-1BB antibody. In some embodiments, the additional therapeutic agent is an anti-PD-1 antibody selected from the groups consisting of: nivolumab (OPDIVO), pembrolizumab (KEYTRUDA), or pidilzumab. In some embodiments, the additional therapeutic agent is an anti-PD-1 antibody selected from the groups consisting of: MEDIO0680, REGN2810, BGB-A317, and PDR001. In some embodiments, the additional therapeutic agent is an anti-PD-L1 antibody selected from the group consisting of: BMS935559 (MDX-1105), atexolizumab (MPDL3280A), durvalumab (MEDI4736), or avelumab (MSB0010718C). In some embodiments, the additional therapeutic agent is an anti-CTLA-4 antibody selected from the group consisting of: ipilimumab (YERVOY) or tremelimumab. In some embodiments, the additional therapeutic agent is an anti-LAG-3 antibody selected from the group consisting of: BMS-986016 and LAG525. In some embodiments, the additional therapeutic agent is an anti-OX-40 antibody selected from the group consisting of: MED16469, MEDI0562, and MOXR0916. In some embodiments, the additional therapeutic agent is an anti-4-1BB antibody selected from the group consisting of: PF-05082566.

Furthermore, treatment with an antineoplastic composition described herein can include combination treatment with biologic molecules, such as one or more cytokines (e.g., lymphokines, interleukins, interferons, tumor necrosis factors, and/or growth factors).

In some embodiments, the antineoplastic agent can be administered in combination with a biologic molecule selected from the group consisting of: adrenomedullin (AM), angiopoietin (Ang), BMPs, BDNF, EGF, erythropoietin (EPO), FGF, GDNF, G-CSF, GM-CSF, GDF9, HGF, HDGF, IGF, migration-stimulating factor, myostatin (GDF-8), NGF, neurotrophins, PDGF, thrombopoietin, TGF-α, TGF-β. TNF-α, VEGF, P1GF, gamma-IFN, IL-1, IL-2, IL-3, IL-4, IL-5, IL-6, IL-7, IL-12, IL-15, and IL-18. In some embodiments, the antineoplastic agent can be administered in combination with a biologic molecule selected from the group consisting of: macrophage colony stimulating factor (M-CSF) and stem cell factor (SCF).

In some embodiments, treatment with an antineoplastic agent described herein can be accompanied by surgical removal of tumors, removal of cancer cells, or any other surgical therapy deemed necessary by a treating physician.

In certain embodiments, treatment involves the administration of an antineoplastic agent of the present invention in combination with radiation therapy. Treatment with an agent can occur prior to, concurrently with, or subsequent to administration of radiation therapy. Dosing schedules for such radiation therapy can be determined by the skilled medical practitioner.

In certain embodiments, treatment involves the administration of an antineoplastic agent in combination with anti-viral therapy. Treatment with an agent can occur prior to, concurrently with, or subsequent to administration of antiviral therapy. The anti-viral drug used in combination therapy will depend upon the virus the subject is infected with.

Combined administration can include co-administration, either in a single pharmaceutical formulation or using separate formulations, or consecutive administration in either order but generally within a time period such that all active agents can exert their biological activities simultaneously.

It will be appreciated that the combination of an antineoplastic agent and at least one additional therapeutic agent may be administered in any order or concurrently. In some embodiments, the agent will be administered to patients that have previously undergone treatment with a second therapeutic agent. In certain other embodiments, the antineoplastic agent and a second therapeutic agent will be administered substantially simultaneously or concurrently. For example, a subject may be given an agent while undergoing a course of treatment with a second therapeutic agent (e.g., chemotherapy). In certain embodiments, an antineoplastic agent will be administered within 1 year of the treatment with a second therapeutic agent. In certain alternative embodiments, an antineoplastic agent will be administered within 10, 8, 6, 4, or 2 months of any treatment with a second therapeutic agent. In certain other embodiments, an antineoplastic agent will be administered within 4, 3, 2, or 1 weeks of any treatment with a second therapeutic agent. In some embodiments, an agent will be administered within 5, 4, 3, 2, or 1 days of any treatment with a second therapeutic agent. It will further be appreciated that the two (or more) agents or treatments may be administered to the subject within a matter of hours or minutes (i.e., substantially simultaneously).

The amount of the therapeutic agents of the invention which will be effective in promoting an antineoplastic effect can be determined by standard clinical techniques. The precise dose to be employed in the formulation will also depend on the judgment of the practitioner and each subject's circumstances. Effective doses can be extrapolated from dose-response curves derived from in vitro or animal model test systems.

One skilled in the art can readily determine an appropriate dosage regimen for administering antineoplastic agents of the invention to a given subject. For example, the compound(s) or composition(s) can be administered to the subject in one administration or multiple administrations. Where a dosage regimen comprises multiple administrations, it is understood that the effective amount of the compound(s) or composition(s) administered to the subject can comprise the total amount of the compound(s) or composition(s) administered over the entire dosage regimen. The exact amount will depend on the purpose of the treatment, the subject to be treated, and will be ascertainable by a person skilled in the art using known methods and techniques for determining effective doses. In some embodiments, the amount of the therapeutic agent that can be administered includes between about 0.1 μg/kg to about 100 mg/kg. In some embodiments, the amount of the therapeutic agent that can be administered includes between about 1.0 μg/kg to about 10 mg/kg.

The antineoplastic agent can be administered one time or over a series of treatments lasting from several days to several months, or until a cure is effected or a diminution of the disease state is achieved (e.g., reduction in tumor size). Optimal dosing schedules can be calculated from measurements of drug accumulation in the body of the patient and will vary depending on the relative potency of an individual agent.

In certain embodiments, dosage is from 0.01 μg to 100 mg/kg of body weight, from 0.1 μg to 100 mg/kg of body weight, from 1 μg to 100 mg/kg of body weight, from 1 mg to 100 mg/kg of body weight, 1 mg to 80 mg/kg of body weight from 10 mg to 100 mg/kg of body weight, from 10 mg to 75 mg/kg of body weight, or from 10 mg to 50 mg/kg of body weight. In certain embodiments, the dosage of the agent is from about 0.1 mg to about 20 mg/kg of body weight. In some embodiments, the dosage of the agent is about 0.5 mg/kg of body weight. In some embodiments, the dosage of the agent is about 1 mg/kg of body weight. In some embodiments, the dosage of the agent is about 1.5 mg/kg of body weight. In some embodiments, the dosage of the agent is about 2 mg/kg of body weight. In some embodiments, the dosage of the agent is about 2.5 mg/kg of body weight. In some embodiments, the dosage of the agent is about 5 mg/kg of body weight. In some embodiments, the dosage of the agent is about 7.5 mg/kg of body weight. In some embodiments, the dosage of the agent is about 10 mg/kg of body weight. In some embodiments, the dosage of the agent is about 12.5 mg/kg of body weight. In some embodiments, the dosage of the agent is about 15 mg/kg of body weight. In certain embodiments, the dosage can be given once or more daily, weekly, monthly, or yearly. In certain embodiments, the agent is given once every week, once every two weeks, once every three weeks, or once every four weeks.

In some embodiments, an antineoplastic agent may be administered at an initial higher “loading” dose, followed by one or more lower doses. In some embodiments, the frequency of administration may also change. In some embodiments, a dosing regimen may comprise administering an initial dose, followed by additional doses (or “maintenance” doses) once a week, once every two weeks, once every three weeks, or once every month. For example, a dosing regimen may comprise administering an initial loading dose, followed by a weekly maintenance dose of, for example, one-half of the initial dose. Or a dosing regimen may comprise administering an initial loading dose, followed by maintenance doses of, for example one-half of the initial dose every other week. Or a dosing regimen may comprise administering three initial doses for 3 weeks, followed by maintenance doses of, for example, the same amount every other week.

As is known to those of skill in the art, administration of any therapeutic agent may lead to side effects and/or toxicities. In some cases, the side effects and/or toxicities are so severe as to preclude administration of the particular agent at a therapeutically effective dose. In some cases, drug therapy must be discontinued, and other agents may be tried. However, many agents in the same therapeutic class often display similar side effects and/or toxicities, meaning that the patient either has to stop therapy, or if possible, suffer from the unpleasant side effects associated with the therapeutic agent.

In some embodiments, the dosing schedule may be limited to a specific number of administrations or “cycles”. In some embodiments, the antineoplastic agent is administered for 3, 4, 5, 6, 7, 8, or more cycles. For example, the agent is administered every 2 weeks for 6 cycles, the agent is administered every 3 weeks for 6 cycles, the agent is administered every 2 weeks for 4 cycles, the agent is administered every 3 weeks for 4 cycles, etc. Dosing schedules can be decided upon and subsequently modified by those skilled in the art.

The present invention provides methods of administering to a subject an antineoplastic agent described herein comprising using an intermittent dosing strategy for administering one or more agents, which may reduce side effects and/or toxicities associated with administration of an agent, chemotherapeutic agent, etc. In some embodiments, a method for treating cancer in a human subject comprises administering to the subject a therapeutically effective dose of an antineoplastic agent in combination with a therapeutically effective dose of a chemotherapeutic agent, wherein one or both of the agents are administered according to an intermittent dosing strategy. In some embodiments, the intermittent dosing strategy comprises administering an initial dose of an antineoplastic agent to the subject, and administering subsequent doses of the agent about once every 2 weeks. In some embodiments, the intermittent dosing strategy comprises administering an initial dose of an antineoplastic agent to the subject, and administering subsequent doses of the agent about once every 3 weeks. In some embodiments, the intermittent dosing strategy comprises administering an initial dose of an antineoplastic agent to the subject, and administering subsequent doses of the agent about once every 4 weeks. In some embodiments, the antineoplastic agent is administered using an intermittent dosing strategy and the chemotherapeutic agent is administered weekly.

In some embodiments, the antineoplastic agents are formulated for microneedle delivery in accordance with routine procedures. In some embodiments, the agents can be formulated in the same manner as pharmaceutical compositions adapted for intravenous, subcutaneous or parenteral administration to human beings. In some embodiments, compositions for administration are solutions in sterile isotonic aqueous buffer. Where necessary, the composition can also include a solubilizing agent. Generally, the ingredients are supplied either separately or mixed together in unit dosage form, for example, as a dry lyophilized powder or water free concentrate in a hermetically sealed container such as an ampoule indicating the quantity of active agent. In some embodiments, an ampoule of sterile water for injection or saline can be provided so that the ingredients can be mixed prior to administration.

The antineoplastic agents can also be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

In certain embodiments, the compositions are pharmaceutical compositions. In some embodiments, formulations are prepared for storage and use by combining the active agents with a pharmaceutically acceptable vehicle (e.g. carrier, excipient) (Remington, The Science and Practice of Pharmacy 20th Edition Mack Publishing, 2000). In some embodiments, pharmaceutical compositions of the present invention are characterized as being at least sterile and pyrogen-free. As used herein, “pharmaceutical formulations” include formulations for human and veterinary use. Pharmaceutical compositions of the invention can be packaged for use in liquid form, or can be lyophilized.

Suitable pharmaceutically acceptable vehicles include, but are not limited to, nontoxic buffers such as phosphate, citrate, and other organic acids; salts such as sodium chloride; antioxidants including ascorbic acid and methionine; preservatives (e.g. octadecyldimethylbenzyl ammonium chloride; hexamethonium chloride; benzalkonium chloride; benzethonium chloride; phenol, butyl or benzyl alcohol; alkyl parabens, such as methyl or propyl paraben; catechol; resorcinol; cyclohexanol; 3-pentanol; and m-cresol); low molecular weight polypeptides (e.g. less than about 10 amino acid residues); proteins such as serum albumin, gelatin, or immunoglobulins; hydrophilic polymers such as polyvinylpyrrolidone; amino acids such as glycine, glutamine, asparagine, histidine, arginine, or lysine; carbohydrates such as monosacchandes, disaccharides, glucose, mannose, or dextrins; chelating agents such as EDTA; sugars such as sucrose, mannitol, trehalose or sorbitol; salt-forming counter-ions such as sodium; metal complexes (e.g. Zn-protein complexes); and non-ionic surfactants such as TWEEN or polyethylene glycol (PEG).

For a broad overview of controlled delivery systems, for example, of polypeptides, see, Banga, A. J., Therapeutic Peptides and Proteins: Formulation, Processing, and Delivery Systems, Technomic Publishing Company, Inc., Lancaster, Pa., (1995). Particulate systems include microspheres, microparticles, microcapsules, nanocapsules, nanospheres, and nanoparticles. Microcapsules can contain the therapeutically active agents as a central core. In microspheres the therapeutic can be dispersed throughout the particle. Particles, microspheres, and microcapsules smaller than about 1 μm are generally referred to as nanoparticles, nanospheres, and nanocapsules, respectively. Microparticles are typically around 100 μm in diameter. See, for example, Kreuter, J., Colloidal Drug Delivery Systems, J. Kreuter, ed., Marcel Dekker, Inc., New York, N.Y., pp. 219-342 (1994); and Tice & Tabibi, Treatise on Controlled Drug Delivery, A. Kydonieus, ed., Marcel Dekker, Inc. New York, N.Y., pp. 315-339, (1992).

In some embodiments, polymers can be used for controlled release of compositions disclosed herein. Various degradable and nondegradable polymeric matrices for use in controlled drug delivery are known in the art (Langer, Accounts Chem. Res. 26:537-542, 1993). For example, the block copolymer, polaxamer 407, exists as a viscous yet mobile liquid at low temperatures but forms a semisolid gel at body temperature. It has been shown to be an effective vehicle for formulation and sustained delivery of recombinant interleukin-2 and urease (Johnston et al., Pharm. Res. 9:425-434, 1992; and Pec et al., J. Parent. Sci. Tech. 44(2):58-65, 1990). Alternatively, hydroxyapatite has been used as a microcarrier for controlled release of proteins (Ijntema et al., Int. J. Pharm. 112:215-224, 1994). In yet another aspect, liposomes can be used for controlled release as well as drug targeting of the lipid-capsulated drug (Betageri et al., Liposome Drug Delivery Systems, Technomic Publishing Co., Inc., Lancaster, Pa. (1993)). Numerous additional systems for controlled delivery of therapeutic proteins are known (see U.S. Pat. Nos. 5,055,303; 5,188,837; 4,235,871; 4,501,728; 4,837,028; 4,957,735; 5,019,369; 5,055,303; 5,514,670; 5,413,797; 5,268,164; 5,004,697; 4,902,505; 5,506,206; 5,271,961; 5,254,342 and 5,534,496).

In some embodiments, the antineoplastic agent described herein can also be entrapped in microcapsules. Such microcapsules are prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nanoparticles and nanocapsules) or in macroemulsions as described in Remington: The Science and Practice of Pharmacy, 22^{st} Edition, 2012, Pharmaceutical Press, London.

In certain embodiments, pharmaceutical formulations include an agent of the present invention complexed with liposomes. Methods to produce liposomes are known to those of skill in the art. For example, some liposomes can be generated by reverse phase evaporation with a lipid composition comprising phosphatidylcholine, cholesterol, and PEG-derivatized phosphatidylethanolamine (PEG-PE). Liposomes can be extruded through filters of defined pore size to yield liposomes with the desired diameter.

In some embodiments, the antineoplastic agent can be formulated in lipid or lipid like nanoparticles.

While the present teachings are described in conjunction with various embodiments, it is not intended that the present teachings be limited to such embodiments. On the contrary, the present teachings encompass various alternatives, modifications, and equivalents, as will be appreciated by those of skill in the art.

Throughout this disclosure, various publications, patents and published patent specifications are referenced by an identifying citation. The disclosures of these publications, patents and published patent specifications are hereby incorporated by reference into the present disclosure to more fully describe the state of the art to which this invention pertains.

1. A method for treating a skin neoplasm in a subject, comprising administering to the subject's skin a composition comprising an effective amount of one or more antineoplastic agents, wherein the composition is administered with a microneedle delivery device.

2. The method of any one of claim 1, wherein the microneedle delivery device comprises
i) one or more microneedles, wherein the microneedles are hollow or non-hollow, wherein one or multiple grooves are inset along an outer wall of the microneedles; and
ii) a reservoir that holds the composition to be delivered, wherein the reservoir is attached to or contains a means to encourage flow of the composition contained in the reservoir into the skin;
wherein the administering comprises a repeated motion of penetrating the microneedle delivery device into the skin of the subject,
wherein the composition is delivered into the skin by passing through the one or multiple grooves along the outer wall of the microneedle.

3. The method of claim 2, wherein the microneedles are non-hollow.

4. The method of claim 1, wherein the means to encourage flow of the composition contained in the reservoir into the skin is selected from the group consisting of a plunger, pump and suction mechanism.

5. The method of claim 4, wherein the means to encourage flow of the composition contained in the reservoir into the skin is a mechanical spring loaded pump system.

6. The method of claim 1, wherein the microneedles have a single groove inset along the outer wall of the microneedle, wherein the single groove has a screw thread shape going clockwise or counterclockwise around the microneedle.

7. The method of claim 1, wherein the microneedles are from 0.1 mm to about 2.5 mm in length and from 0.01 mm to about 0.05 mm in diameter.

8. The method of claim 1, wherein the microneedles are made from a substance comprising gold.

9. The method of claim 2, wherein the plurality of microneedles comprises an array of microneedles in the shape of a circle.

10. The method of claim 1, wherein the microneedles are made of 24-carat gold plated stainless steel and comprise an array of 20 microneedles.

11. The method of claim 1, wherein the antineoplastic agent is selected from the group consisting of alkylating agents, platinum compounds (e.g., cisplatin, carboplatin, oxaliplatin, mechlorethamine, cyclophosphamide, chlorambucil, ifosfamide), anti-metabolic agents (e.g., purine and pyrimidine analogues, antifolates), anthracyclines (doxorubicin, daunorubicin, valrubicin, idarubicin, epirubicin), cytotoxic antibiotics (actinomycin, bleomycin, plicamycin, mitomycin), monoclonal antibodies (e.g., Alemtuzumab, Bevacizumab, Cetuximab, Gemtuzumab, Ibritumomab, Panitumumab, Rituximab, Tositumomab, and Trastuzumab), kinase inhibitors (e.g., imatinib, erlotinib, gefitinib), plant alkaloids and terpenoids, topoisomerase inhibitors (e.g., camptothecins, irinotecan, topotecan, amsacrine, etoposide, etoposide phosphate, teniposide), vinca alkaloids (e.g., vincristine, vinblastine, vinorelbine, vindesine), taxanes (e.g., paclitaxel, taxol, docetaxel), podophyllotoxins, epipodophyllotoxins and combinations thereof.

12. The method of claim 1, wherein the skin neoplasm is selected from the group consisting of keratinocytic neoplasms (such as basal cell carcinoma, squamous cell carcinoma, Bowen's disease, bowenoid papulosis, Merkel cell carcinoma, actinic keratosis, and keratoacanthoma), melanocytic neoplasms (including all types of melanoma, such as superficial spreading melanoma, nodular melanoma, lentigo melanoma, acral-lentiginous melanoma, desmoplastic melanoma, nevoid melanoma, and amelanotic melanoma), appendageal neoplasms, soft tissue neoplasms, neural neoplasms, and cutaneous neoplasms.

13. The method of claim 1, wherein the skin neoplasm is benign, pre-malignant, malignant, or a metastatic skin neoplasms.

14. The method of claim 1, further comprising administering to the subject one or more additional therapies.

15. The method of claim 14, wherein the additional therapy includes radiation, surgery, chemotherapy, simple excision, Mohs micrographic surgery, Curettage and electrodesiccation, cryosurgery, photodynamic therapy, topical chemotherapy, topical immunotherapy, intravenously administered therapeutic agent, and orally administered therapeutic agent.

16. A microneedle drug delivery device comprising a composition comprising an effective amount of one or more antineoplastic agents.

17. The microneedle drug delivery device of claim 16, wherein the antineoplastic agent is selected from the group consisting of alkylating agents, platinum compounds (e.g., cisplatin, carboplatin, oxaliplatin, mechlorethamine, cyclophosphamide, chlorambucil, ifosfamide), anti-metabolic agents (e.g., purine and pyrimidine analogues, antifolates), anthracyclines (doxorubicin, daunorubicin, valrubicin, idarubicin, epirubicin), cytotoxic antibiotics (actinomycin, bleomycin, plicamycin, mitomycin), monoclonal antibodies (e.g., Alemtuzumab, Bevacizumab, Cetuximab, Gemtuzumab, Ibritumomab, Panitumumab, Rituximab, Tositumomab, and Trastuzumab), kinase inhibitors (e.g., imatinib, erlotinib, gefitinib), plant alkaloids and terpenoids, topoisomerase inhibitors (e.g., camptothecins, irinotecan, topotecan, amsacrine, etoposide, etoposide phosphate, teniposide), vinca alkaloids (e.g., vincristine, vinblastine, vinorelbine, vindesine), taxanes (e.g., paclitaxel, taxol, docetaxel), podophyllotoxins, epipodophyllotoxins and combinations thereof.

18. The microneedle drug delivery device of claim 16, wherein the antineoplastic agent is in lyophilized or powder form.
